(19) United States
(12) Patent Application Publication
(10) Pub. No.: US 2024/0412383 A1
(43) Pub. Date: Dec. 12, 2024

(54) SYSTEMS AND METHODS FOR GENERATING A BIOMECHANICAL MODEL OF A SUBJECT IN MOTION

(71) Applicant: VueMotion Labs Pty Ltd., Sutherland, New South Wales (AU)

(72) Inventors: Amit Gupta, Sutherland, New South Wales (AU); David Klineberg, Sutherland, New South Wales (AU); Ryan Talbot, Sutherland, New South Wales (AU)

(21) Appl. No.: 18/699,218

(22) PCT Filed: Oct. 7, 2022
(86) PCT No.: PCT/AU2022/051206

§ 371 Date: Apr. 5, 2024

(30) Foreign Application Priority Data
Oct. 7, 2021 (AU) 2021903222
Oct. 7, 2021 (AU) 2021903223
Oct. 7, 2021 (AU) 2021903224

(51) Int. Cl. G06T 7/246; G06T 7/73; G06V 40/20

(52) U.S. Cl. CPC G06T 7/246; G06T 7/73; G06V 40/25; G06T 2207/10016; G06T 2207/30196; G06T 2207/30221

(57) ABSTRACT

There is provided a system for generating a motion performance metric of a moving human subject. The system includes a single stationarily supported motion capture device in the form of a smartphone having a camera configured to capture from a predetermined capture position, visual data of the subject as the subject moves (for example, by walking, jogging and/or running) between two distance calibration markers that are disposed at a predetermined distance apart from each other and in a field of vision of the camera. The system further includes a central data processing server in communication with the smartphone. The server is configured to initially recognise, from the captured visual data, a plurality of human pose points on the subject. The server then is able to extract kinematic data of the subject based on the recognised human pose points and then subsequently construct, based on the extracted kinematic data, a biomechanical model of the motion of the subject. The server then formulates a motion performance metric based on the constructed biomechanical model.

### TECHNICAL FIELD

The present disclosure relates to systems and methods for generating a biomechanical model of a subject in motion. The present disclosure has applications to sports science and in particular to analysis of the physical movement of a subject and related performance based on the subject movement.

While some embodiments will be described herein with particular reference to that application, it will be appreciated that the invention is not limited to such a field of use, and is applicable in broader contexts.

### BACKGROUND

Any discussion of the background art throughout the specification should in no way be considered as an admission that such art is widely known or forms part of common general knowledge in the field.

In the realm of competitive sports, there has been a desire to adapt and improve training techniques in order to increase performance. In the digital age, the use of technology, including specific devices, to track and analyse performance in certain sports is becoming very widely used. Initially, these were only basic tools such as stop watches, pedometers and heart rate monitors which provide a result of the performance of an athlete, but not any analysis of the physical form or pose of an athlete.

Further, such technology was traditionally only utilised by those at a professional level of their sport due to the expense and complexity involved. However, in more recent times, the costs and complexities have decreased significantly whereby the use of forms of performance tracking and analysis technology is prevalent amongst amateur sports enthusiasts. Sports performance tracking and analysis often requires one or more specific wearable device. Such devices can include heartrate monitors, motion sensors, location trackers (such as GPS) and specific garments that have trackable markers. One significant disadvantage with known wearable sensor devices is the inherent requirement of the device to be worn and fitted to an athlete. Such a requirement burdens the athlete with additional load and inconvenience and also changes the natural condition of activity in that such wearable devices would otherwise not be worn to perform the athletic activity. Further, wearable devices can be restrictive in true natural motion of the athlete and do not represent the full context of activity including, for example, environmental conditions and surface type, amongst others.

The type of device used will often be tailored to a specific sport and the equipment used and movements involved in that sport. For example, a specific hardware device fitted in a soccer ball to measure the speed and motion profile of the soccer ball, or a cyclist may have a specific device that is fitted to their bicycle to measure cadence, that would not be used for other sports.

As such, many present day performance tracking and analysis technologies are limited by their requirement of specific hardware devices, for example specifically designed cameras, and further limited to their reliance on the activity being captured in a controlled environment, for example the use of specific sensors to record human movement such as cameras with depth sensor and/or a treadmill for person running.

Further, the data collected by such devices must be analysed in order to extract any meaningful information for the athlete. This analysis and the results from the analysis was initially done for professional athletes by sports scientists in a specific controlled environment such as an indoor gait laboratory. The requirements of specific environmental conditions are restrictive towards high frequency regular analysis and data. Additionally, quite often the analysis and data outputs vary between lab environment and natural environment. However, more recently, some performance metrics of varying levels of usefulness have been automated through the abovementioned technologies. Further, the actual presentation of the performance tracking and analysis has an extremely significant bearing on the usefulness of that information to the athlete.

Known systems and devices also generally to do not suggest any insights for the athlete to improve their performance, as these are left to coaches, sports scientists, or the athlete themselves to deduce.

It will also be appreciated that known systems are generally not capable of capturing the motion of more than one athlete at a time.

### SUMMARY

It is an object of the present invention to overcome or ameliorate at least one of the disadvantages of the prior art, or to provide a useful alternative.

In accordance with a first aspect of the present invention there is provided a method for generating a motion performance metric including the steps of:

capturing, by a single supported motion capture device from a capture position, visual data of a subject as it moves between at least two distance markers in a field of vision of the motion capture device;
from the captured visual data, extracting kinematic data of the subject; and
based on the extracted kinematic data, formulating a motion performance metric.
In an embodiment, the at least two distance markers that are disposed at a predetermined distance from each other.

In an embodiment, extracting kinematic data of the subject includes recognising human pose points on the subject.

In an embodiment, the method includes the further step of: constructing a biomechanical model of the motion of the subject based on the extracted kinematic data, whereby the motion performance metric is formulated based on the constructed biomechanical model.

In an embodiment, the motion capture device is substantially stationarily supported.

In an embodiment, the motion capture device is a camera. In an embodiment, the camera is a smartphone camera. In another embodiment, the camera is an IP camera.

In an embodiment, the motion capture device includes two synchronised cameras.

In an embodiment, the visual data of a subject is captured without the use of wearable subject makers on the subject.

In an embodiment, the motion performance metric includes one or more of: velocity of the subject; stride length of the subject; stride frequency of the subject; and form of the subject.

In an embodiment, a plurality of motion performance metrics is formulated.

In an embodiment, the method includes the further step of outputting the motion performance metric for visual display on a display device.

In an embodiment, the display device is a smartphone.

In an embodiment, the motion performance metric is outputted and displayed as one or more of: a graph; a number; and a dynamically moving gauge.

In an embodiment, the subject is captured as it moves between two distance markers, the two distance markers being disposed at a predetermined distance of 20 metres from each other.

In accordance with a second aspect of the present invention there is provided a system for generating a motion performance metric including:
a single supported motion capture device configured to capture from a capture position, visual data of a subject as it moves between at least two distance markers in a field of vision of the motion capture device; and
a central data processing server in communication with the motion capture device, the central data processing server configured to:
   extract, from the captured visual data, kinematic data of the subject; and
   formulate a motion performance metric based on the extracted kinematic data.

In accordance with a third aspect of the present invention there is provided a method for generating a biomechanical model of a subject in motion including the steps of:
capturing, by a single supported motion capture device from a capture position, visual data of the subject as it moves between two distance markers that are disposed at a distance apart from each other and in a field of vision of the motion capture device;
from the captured visual data, recognising human pose points on the subject to extract kinematic data of the subject; and
based on the extracted kinematic data, recognising a plurality of predefined anatomical components of the subject to construct the biomechanical model of the subject.

In an embodiment, the captured visual data includes a plurality of video frames, and the constructed biomechanical model is based on at least one frame capturing the subject in a predefined stance.

In an embodiment, the predefined stance is one or more of:
a toe-off stance whereby a back foot of the subject is lifted off a ground push-off point;
a touch down stance whereby a front foot of the subject is about to contact a ground drop point; and
a full support stance whereby the front foot flattens on the ground drop point and whereby hip and heel human pose points of the subject are vertically aligned.

In an embodiment, the motion capture device is substantially stationarily supported.

In an embodiment, the motion capture device is a camera. In an embodiment, the camera is a smartphone camera. In another embodiment, the camera is an IP camera.

In an embodiment, the motion capture device includes two synchronised cameras.

In an embodiment, the visual data of a subject is captured without the use of wearable subject makers on the subject.

In an embodiment, the distance between the two distance markers is a predetermined distance of 20 metres.

In an embodiment, the method includes the further step of formulating based on the constructed biomechanical model, a motion performance metric.

In an embodiment, the method includes the further step of outputting the motion performance metric for visual display on a display device.

In accordance with a fourth aspect of the present invention there is provided a system for generating a biomechanical model of a subject in motion including:
a single supported motion capture device configured to capture from a capture position, visual data of a subject as it moves between two distance markers that are disposed at a distance apart from each other and in a field of vision of the motion capture device;
a central data processing server in communication with the motion capture device, the central data processing server configured to:
   recognise, from the captured visual data, human pose points on the subject;
   extract, from the recognised human pose points, kinematic data of the subject;
   recognise, based on the extracted kinematic data, a plurality of predefined anatomical components of the subject; and
   construct, based on the recognised plurality of predefined anatomical components, the biomechanical model of the subject.

In accordance with a fifth aspect of the present invention there is provided a method for providing motion performance feedback to a subject, the method including the steps of:
capturing, by a single supported motion capture device from a capture position, visual data of the subject as it moves between at least two distance markers in a field of vision of the motion capture device;
from the captured visual data, extracting kinematic data of the subject;
based on the extracted kinematic data, formulating a motion performance metric; and
generating a target motion performance metric based on the formulated motion performance metric, such that the target motion performance metric represents a predefined improvement increment over the formulated motion performance metric; and
generating motion performance feedback to be provided to the subject, the motion performance feedback based on the difference between the target motion performance metric and the formulated motion performance metric.

In an embodiment, the formulated motion performance metric includes current stride frequency and the target motion performance metric includes a target stride frequency.

In an embodiment, the predefined improvement increment is 1%, such that the target stride frequency is 1% faster than the current stride frequency.

In an embodiment, the motion performance feedback includes providing an audible cue to the subject at a frequency that is equivalent to the target stride frequency.

In an embodiment, the method includes the further step of: providing a wearable audio output device to the subject from which the audible cue is outputted and heard by the subject.

In an embodiment, the motion capture device is substantially stationarily supported.

In an embodiment, the motion capture device is a camera. In an embodiment, the camera is a smartphone camera. In another embodiment, the camera is an IP camera.

In an embodiment, the motion capture device includes two synchronised cameras.

In an embodiment, the distance between the two distance markers is a predetermined distance of 20 metres.

In accordance with a sixth aspect of the present invention there is provided a system for providing motion performance feedback to a subject, the system including:
a single supported motion capture device configured to capture from a capture position, visual data of the subject as it moves between two distance markers that are disposed at a distance apart from each other and in a field of vision of the motion capture device;
a central data processing server in communication with the motion capture device, the central data processing server configured to:
   extract, from the captured visual data, kinematic data of the subject;
   formulate, based on the extracted kinematic data, a motion performance metric;
   generate, based on the formulated motion performance metric, a target motion performance metric such that the target motion performance metric represents a predefined improvement increment over the formulated motion performance metric; and
   generate motion performance feedback to be provided to the subject, the motion performance feedback based on the difference between the target motion performance metric and the formulated motion performance metric.

In accordance with a seventh aspect of the present invention there is provided a method for generating a motion performance metric including the steps of:
capturing, by a single stationarily supported motion capture device from a predetermined capture position, visual data of a moving subject in a field of vision of the motion capture device, the subject including a component having a known real-world length;
from the captured visual data, recognising human pose points on the subject to extract kinematic data of the subject, including a captured length of the component of the subject;
mapping the known real-world length of the component to the captured length of the component;
based on the extracted kinematic data, constructing a biomechanical model of the motion of the subject whereby the biomechanical model includes real-world lengths based on the mapping; and
based on the biomechanical model, formulating a motion performance metric.

In accordance with an eighth aspect of the present invention there is provided a method for generating a biomechanical model of at least two subjects in motion including the steps of:
capturing, by a single stationarily supported motion capture device from a predetermined capture position, visual data of the at least two subjects as they move between two distance markers that are disposed at a predetermined distance apart from each other and in a field of vision of the motion capture device;
from the captured visual data, individually detecting each of the at least two subjects such that each detected subject is isolated;
from the captured visual data, for each detected subject:
   recognising human pose points on the subject to extract kinematic data of the subject; and
   based on the extracted kinematic data, recognising a plurality of predefined anatomical components of the subject to construct the biomechanical model of the subject.

In accordance with a ninth aspect of the present invention there is provided a method for providing a visual comparison of a first biomechanical model and a second biomechanical model, the method including the steps of:
generating the first and second biomechanical models according to the method of the third aspect;
identify corresponding central points of reference on each of the first and second biomechanical models;
scale each of the first and second biomechanical models such that relative heights of the first and second biomechanical models are identical; and
overlay the scaled first and second biomechanical models at their respective corresponding central points of reference to provide the visual comparison of the first and second biomechanical models.

In accordance with a tenth aspect of the present invention there is provided a method for identifying a current subject based on a predefined limb length ratio of a known subject, the method including the steps of:
generating a biomechanical model of the current subject in motion according to the method of the third aspect, wherein the plurality of predefined anatomical components of the current subject includes a first subject limb and a second subject limb of the subject, the first subject limb having a first limb length and the second subject limb having a second limb length;
generating a current limb length ratio based on the first limb length and the second limb length;
comparing the current limb length ratio to the predefined limb length ratio; and
if the current limb length ratio and the predefined limb length ratio substantially matches, identifying the current subject as being the known subject.

Other aspects of the present disclosure are also provided.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may be in some appropriate cases. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

### BRIEF DESCRIPTION OF THE DRAWINGS

One or more embodiments of the present disclosure will now be described by way of specific example(s) with reference to the accompanying drawings, in which:

FIG. 1 is a schematic diagram of a system for generating a motion performance metric according to an embodiment of the invention;

FIG. 2 is a block diagram of a computing system with which various embodiments of the present disclosure can be implemented/configurable to perform various features of the present disclosure;

FIGS. 3A to 3C are representations of the subject captured by a camera of the system of FIG. 1;

FIG. 3D is a representation of an embodiment of an estimated human pose model shown annotated in FIG. 3C;

FIG. 4 is a representation of an embodiment of a biomechanical model constructed by the system of FIG. 1;

FIG. 5A is a representation of an embodiment of anatomical pose points estimated by a human pose estimation model formed by the system of FIG. 1;

FIG. 5B is an enlarged representation of an embodiment of anatomical pose points showing the feet, as estimated by a human pose estimation model;

FIG. 5C is an enlarged representation of an embodiment of anatomical pose points showing the knees, as estimated by a human pose estimation model;

FIG. 5D is an enlarged representation of an embodiment of anatomical pose points showing the hips, as estimated by a human pose estimation model;

FIG. 5E is an enlarged representation of an embodiment of anatomical pose points showing the elbows, as estimated by a human pose estimation model;

FIG. 5F is an enlarged representation of an embodiment of anatomical pose points showing the shoulders, as estimated by a human pose estimation model;

FIG. 6 is a representation of a triple point crossover joint system of the human pose estimation model of FIG. 5A;

FIG. 7A is a set of photographic stills of a subject captured by a camera of the system of FIG. 1;

FIG. 7B are representations of a set of estimated human pose models corresponding to the photographic stills of FIG. 7A;

FIG. 7C are representations of a set of biomechanical models corresponding to the photographic stills of FIG. 7A and the estimated human pose models of FIG. 7B;

FIG. 8 is a series of representations of an embodiment of a biomechanical model showing an overlapping comparison of models;

FIG. 9 is a representation of an embodiment of a limb ratio chart generated by the system of FIG. 1 based on a human pose estimation model;

FIG. 10 is an alternate schematic representation of the system of FIG. 1;

FIG. 11 is a plot of stride points of a subject captured by a camera of the system of FIG. 1;

FIG. 12 is a flow chart of the process of using the system of FIG. 1;

FIGS. 13A to 13C are plots of stride points similar to FIG. 11 each showing different movement scenarios;

FIGS. 14A and 14B are representations of embodiments of plots of stride points similar to FIGS. 11 and 13A to 13C;

FIGS. 14C and 14D are representations of embodiments of a performance metric;

FIGS. 15A and 15B are representations of embodiments of estimated human pose models shown with plots of stride points similar to FIGS. 11, 13A to 13C, 14A and 14B;

FIGS. 16A and 16B are representations of embodiments of plots of stride points similar to FIGS. 11, 13A to 13C, 14A, 14B, 15A and 15B;

FIG. 17 is a representation of an embodiment of a plurality of performance metrics;

FIG. 18 is an embodiment of a webpage;

FIGS. 19A to 19D are representations of embodiments of a plurality displayed performance metrics;

FIG. 20 is a representation of an embodiment of a plurality displayed performance metrics;

FIG. 21 is a representation of an embodiment of a plurality displayed performance metrics;

FIG. 22 is a schematic diagram of an alternate embodiment of the system of FIG. 1;

FIG. 23 is a composite representation of a subject captured by a camera of the system of FIG. 1 showing in a single representation the position of the subject at two frames;

FIG. 24A is line graph representation of the position of the left heel pose point of a subject captured by a camera of the system of FIG. 1;

FIG. 24B is line graph representation of the difference in position of the left heel pose point of a subject captured by a camera of the system of FIG. 1;

FIG. 25 is composite line graph representation of the position of two comparative pose points of a subject captured by a camera of the system of FIG. 1;

FIG. 26 are representations of two sets of estimated human pose models capturing a subject during an acceleration motion, the first corresponding to a “toe-off” pose and the second corresponding to a “touch down” pose both;

FIG. 27A are representations of two sets of estimated human pose models capturing a subject during a run motion, the first corresponding to a plurality of phase poses in respect of the left foot and the second corresponding to a plurality of phase poses in respect of the right foot; and

FIG. 27B is an enlarged view of the toe off phase pose of in respect of the right foot shown in FIG. 27A.

### DETAILED DESCRIPTION

Where applicable, steps or features in the accompanying drawings that have the same reference numerals are to be considered to have the same function(s) or operation(s), unless the contrary intention is expressed or implied.

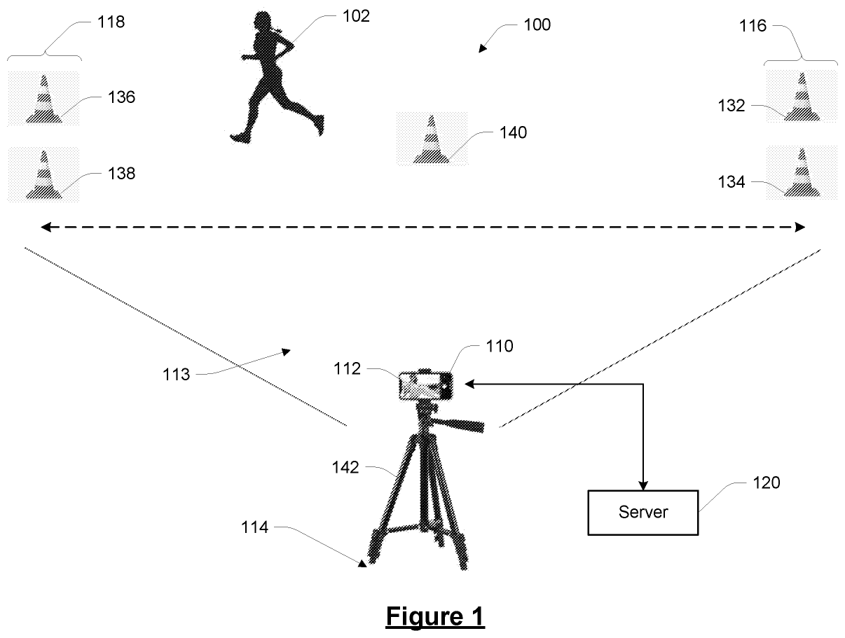

Referring initially to FIG. 1, there is illustrated a system 100 for generating a motion performance metric of a moving human subject 102. System 100 includes a single stationarily supported motion capture device in the form of a smartphone 110 having a camera 112 configured to capture from a predetermined capture position 114, visual data of subject 102 as subject 102 moves (for example, by walking, jogging and/or running) between two distance calibration markers 116 and 118 that are disposed at a predetermined distance of 20 metres apart from each other and in a field of vision 113 of camera 112.

System 100 further includes a central data processing server 120 in communication with smartphone 110. Server 120 is configured to initially recognise, from the captured visual data, a plurality of human pose points on subject 102. Server 120 then is able to extract kinematic data of subject 102 based on the recognised human pose points and then subsequently construct, based on the extracted kinematic data, a biomechanical model of the motion of subject 102. Finally, server 120 formulates a motion performance metric based on the constructed biomechanical model.

Distance markers 116 and 118 each include a pair of recognisable objects, in this embodiment a pair of marker cones, where distance marker 116 includes marker cones 132 and 134, and distance marker 118 includes marker cones 136 and 138. Each respective pair of marker cones are placed at a predetermined marker distance apart, that distance being approximately 1.2 metres such that the four cones form a rectangular area of 1.2 metres by 20 metres. Intermediate distance markers 116 and 118 there is placed a centre marker cone 140 that will be directly in the centre of the rectangular area, 10 metres from each of distance markers 116 and 118 for marking the centre point of the 20 metre interval between distance markers 116 and 118. It will be appreciated that the use of centre marker cone 140 facilitates more accurate modelling, in terms of mapping the captured visual data to the real-world distances. In some embodiments, centre marker cone 140 is not utilised with only marker cones 132, 134, 136 and 138 being used. In other embodiments, physical markers other than marker cones are used, for example vertically mounted stakes. It will also be appreciated that, in other embodiments, each respective pair of marker cones are placed at a predetermined marker distance apart that is more or less than approximately 1.2 metres. In other embodiments, each of distance markers 116 and 118 each include only a single marker cone. Further, whilst marker cones are utilised in preferred embodiments, system 100 is such that any identifiable physical marker can be used to mark the 20 metre interval.

Camera 112 is stationarily mounted on a tripod 142 at capture position 114 in order to capture subject 102 in respect of its sagittal plane (that is, from a “side on” perspective). As noted above, capture position 114 is such that distance markers 116 and 118 are in field of vision 113 of camera 112 and such that both distance markers 116 and 118 can been seen by camera 112 whilst stationary and, therefore, the entire 20 metre interval can be seen without having to move camera 112. Further, capture position 114 is as close as possible to the 20 metre interval such that the 20 metre interval fills a significant part of the width of field of vision 113. Additionally, capture position 114 is approximately centred in respect of distance markers 116 and 118, such that capture position 114 is approximately equidistant from each of distance markers 116 and 118. It will be appreciated that camera 112, in other embodiments, is mounted to a structure other than tripod 142 or is simply held stationary by a person during the visual data capture of subject 102 moving between distance markers 116 and 118.

In alternate embodiments, system 100 has a setup using two of cameras 112 that are synchronized such that a subject will be measured moving over a 40 metre interval. In this embodiment, each of the cameras are configured to capture visual data of subject 102 whereby one camera is setup in its predetermined capture position such that it captures the subject moving over one 20 metre interval and the other camera is setup in its predetermined capture position such that it captures the subject moving over the other 20 metre interval. Between the two cameras, this configuration is such that subject 102 is captured moving over the full 40 metre interval. In further embodiments, system 100 will have more than two cameras 112 such that each camera is similarly set up to capture the subject moving over its respective 20 metre interval.

Referring to FIG. 22, in another embodiment, a second camera 2212 (in this embodiment another smart phone 2210) can be used synchronized to the first reference camera, camera 2212 having a field of view 2213. Cameras 112 and 2212 are configured to record at a same frame rate, for example a frame rate of 60 frames per second is used. Cameras 112 and 2212 are synchronized by using an action event which is detected in both cameras, for example a “toe-off” pose closest to distance marker 116 (either ahead or behind) in both the camera views. In another embodiment, two cameras are synchronised using a common clock. Camera 2212 is focuses on subject 102 such that field of view 2213 includes subject 102, but may not include each of cones 132, 134, 136 and 138, along with centre marker cone 140. Camera 2212 focuses on subject 102 as it moves within the 20 metre interval, following and keeping focus of subject 102. As such, camera 2212 is used to capture a more zoomed-in version of subject 102 for higher signal to noise ratio, this ratio being defined by number of pixels representing the subject (the “signal”) versus total pixels. In another embodiment, marker cones 132, 134, 136 and 138, along with centre marker cone 140 are in both fields of view 113 and 2213, where the markers cones are used as reference points along with the point at which the left foot of subject 102 touches the ground nearest to the marker cones. It is noted that the second camera does not have to be static.

Camera 112 is a standard built in camera on an off-the-shelf smartphone 110. The captured visual data will be in the form of a 2D video having a plurality of frames. Camera 112 provides a video output with a certain frame rate and resolution, for example such as 4K resolution at 60 frames per second. It will be appreciated that a camera resolution of at least 1080p is required based on the 20 metre interval distance. Such a resolution will allow the captured visual data to be of a quality where the necessary human pose points of subject 102 can be clearly extracted, along with the automatic detection of marker cones 132, 134, 136 and 138. In other embodiments, the camera 112 is other than a smartphone camera, for example an IP camera or other standard camera having the required resolution and capability of communicating with server 120.

In other embodiments, an interval length for a single camera will be other than 20 metres, with the interval length depending on the resolution capabilities of camera 112. For example, for a camera that can capture video at a very high resolution, the interval length able to be greater than 20 metres in order for the captured visual data to be of sufficient quality to recognise the requisite visual markers and points. It will be appreciated that system 100 will be configured to analyse movement of subject 102 between any predefined known interval length using the same techniques as described herein.

Smartphone 110 includes a dedicated software application for receiving the captured visual data and transmitting that visual data to server 120 for processing and analysis. In preferred embodiments, the dedicated application is able to access the camera functionality of smartphone 110 and control camera 112 such that field of vision 113 of camera 112 is shown within the dedicated application whilst it is currently running in the foreground. In this case, the dedicated application can be said to actually capture the visual data. The dedicated application will include controls for a user to activate and deactivate the data capture functionality of camera 112 in order to capture the movement of subject 102 in the form of a video. The dedicated application will then prompt the user to upload the captured visual data to server 120. Further, the dedicated application also receives the formulated motion performance metric that is outputted from server 120 for visual display by smartphone 110. It will be appreciated that smartphone 110 will also have other applications installed on it and running thereon, for example an operating system. In other embodiments, system 100 includes an alternate display device for visually displaying the formulated motion performance metric that is outputted from server 120, for example a separate laptop computer, tablet or different smartphone. In other alternate embodiments, camera 112 coupled to a desktop or laptop computer. It will be appreciated that, in other embodiments, other appropriate computing devices are utilized such as a tablet computer or PDA.

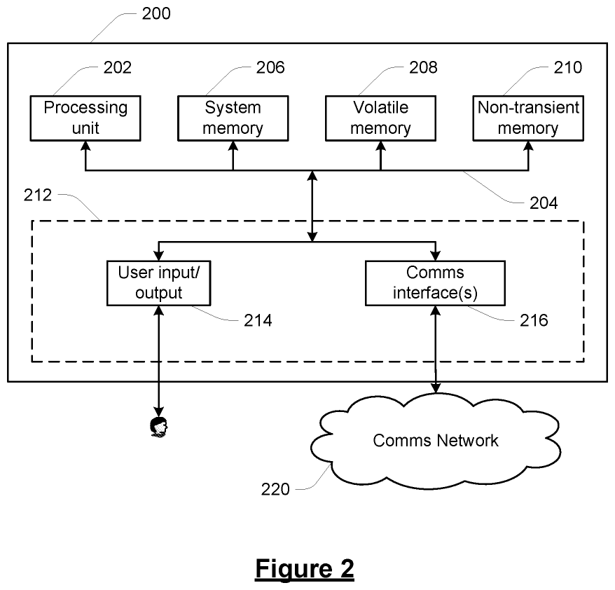

FIG. 2 provides a block diagram of a computer processing system 200 configurable to perform various functions described herein, for example the functions of smartphone 110. System 200 will be described below essentially as a general purpose computer processing system that includes the elements that exist in smartphone 110 along with elements for other computing devices. It will be appreciated that FIG. 2 does not illustrate all functional or physical components of a computer processing system. For example, no power supply or power supply interface has been depicted. However, system 200 will either carry a power supply or be configured for connection to a power supply (or both). It will also be appreciated that the particular type of computer processing system will determine the appropriate hardware and architecture, and in some embodiments alternative computer processing systems suitable for implementing features of the present disclosure will have additional, alternative, or fewer components than those depicted.

Computer processing system 200 includes at least one processing unit 202. In some embodiments, processing unit 202 is a single computer processing device (for example, a central processing unit, graphics processing unit, or other computational device). In other embodiments, processing unit 202 includes a plurality of computer processing devices. In some embodiments, where system 200 is described as performing an operation or function, all processing required to perform that operation or function will be performed by processing unit 202. In other embodiments, processing required to perform that operation or function is also performed by remote processing devices accessible to and useable by (either in a shared or dedicated manner) system 200, such as server 120.

Through a communications bus 204, processing unit 202 is in data communication with a one or more machine readable storage (memory) devices which store instructions and/or data for controlling operation of system 200. In various embodiments, system 200 includes one or more of: a system memory 206 (for example, resident set-size memory), volatile memory 208 (for example, random access memory), and non-volatile or non-transitory memory 210 (for example, one or more hard disk or solid-state drives). Such memory devices may also be referred to as computer readable storage media.

System 200 also includes one or more interfaces, indicated generally by reference 212, via which system 200 interfaces with various devices and/or networks. Generally speaking, in various embodiments, other devices are integral with system 200, or are separate. Where a device is separate from system 200, connection between the device and system 200, in various embodiments, is via wired or wireless hardware and communication protocols, and are a direct or an indirect (for example, networked) connection.

Wired connection with other devices/networks is facilitated by any appropriate standard or proprietary hardware and connectivity protocols. For example, in various embodiments, system 200 is be configured for wired connection with other devices/communications networks by one or more of: USB; FireWire; Ethernet; HDMI; and other wired connection interfaces.

Wireless connection with other devices/networks is similarly facilitated by any appropriate standard or proprietary hardware and communications protocols. For example, in various embodiments, system 200 is configured for wireless connection with other devices/communications networks using one or more of: infrared; Bluetooth; Wi-Fi; near field communications (NFC); Global System for Mobile Communications (GSM); Enhanced Data GSM Environment (EDGE); long term evolution (LTE); and other wireless connection protocols.

Generally speaking, and depending on the particular system in question, devices to which system 200 connects (whether by wired or wireless means) include one or more input devices to allow data to be input into/received by system 200 for processing by processing unit 202, and one or more output device to allow data to be output by system 200. A number of example devices are described below. However, it will be appreciated that, in various embodiments, not all computer processing systems will include all mentioned devices, and that additional and alternative devices to those mentioned are used.

Referring to reference 214, in one embodiment, system 200 includes (or connects to) one or more input devices by which information/data is input into (received by) system 200. Such input devices include keyboards, mice, trackpads, microphones, accelerometers, proximity sensors, GPS devices and the like. System 200, in various embodiments, further includes or connects to one or more output devices controlled by system 200 to output information. Such output devices include devices such as a cathode ray tube (CRT) displays, liquid-crystal displays (LCDs), light-emitting diode (LED) displays, plasma displays, touch screen displays, speakers, vibration modules, LEDs/other lights, amongst others. In preferred embodiments, system 200 includes or connects to devices which are able to act as both input and output devices, for example memory devices (hard drives, solid state drives, disk drives, compact flash cards, SD cards and the like) which system 200 can read data from and/or write data to, and touch screen displays which can both display (output) data and receive touch signals (input).

System 200 also includes one or more communications interfaces 216 for communication with a network 220. Via the communications interface(s) 216, system 200 can communicate data to and receive data from networked devices, which in some embodiments are themselves other computer processing systems.

System 200 stores or has access to computer applications (also referred to as software, applications or programs), such as the dedicated application. These are also described as computer readable instructions and data which, when executed by the processing unit 202, configure system 200 to receive, process, and output data. Instructions and data are able to be stored on non-transient machine readable medium accessible to system 200. For example, in an embodiment, instructions and data are stored on non-transient memory 210. Instructions and data are able to be transmitted to/received by system 200 via a data signal in a transmission channel enabled (for example) by a wired or wireless network connection over interface such as 212.

Applications accessible to system 200 typically includes an operating system application such as Windows, macOS, IOS, Android, Unix, Linux, or other operating system.

In respect of the relationship between smartphone 110 and server 120, in terms of architecture, the communications and interactions generally reflect a client/server relationship whereby smartphone 110 is a client-side device and server 120 is a server side device.

When executed by smartphone 110 (for example, by a processing unit such as 202), the dedicated application configures smartphone 110 to provide client-side visual data capture and display functionality. This involves communicating (using network 220) with server 120. In embodiments, the dedicated application communicates with sever 120 using an application programming interface (API). Alternatively, in other embodiments, the application is a web browser (such as Chrome, Safari, Internet Explorer, Firefox, or an alternative web browser) which communicates with a web server of sever 120 (or server 120 itself being a web server) using http/https protocols over network 220, https protocols being known as encrypted web traffic.

Furthermore, while a smartphone 110 has been depicted, system 100 will typically include multiple smartphones 110, each configured in a similar fashion to interact with server 120. Further, server 120 is configured to provide server-side functionality for each of the end users by way of the one or multiple smartphones 110, by receiving and responding to requests from the one or multiple smartphones 110. In embodiments where the application is a web browser, server 120 includes a web server (for interacting with the web browser clients). Otherwise, server 120 includes an application server (such as a network available applications service including a service providing API using web protocols, for example, http/https or gRPC) for interacting with dedicated application clients by way of the dedicated application. While server 120 has been illustrated as a single server, in other embodiments, server 120 consists of multiple servers (for example, one or more web servers and/or one or more application servers).

Server 120 preferably takes the form of a cloud based server-side computer that will naturally include hardware such as a processor and memory as well as software that is executable by the hardware. Server 120 includes one or more cloud-based databases for storing information including: user profile information for each subject 102; raw visual data; kinematic data; biomechanical model data; and motion performance metric data, amongst others. In other embodiments, server 120 is a locally hosted server-side computer and associated database.

In other embodiments, smartphone 110 itself will include the required functionality of server 120 such that server 120 is not necessary as all data processing including the broad steps (described in greater detail below) of: recognising, from the captured visual data, the plurality of human pose points on subject 102; extracting kinematic data of subject 102 based on the recognised human pose points; constructing, based on the extracted kinematic data, the biomechanical model of the motion of subject 102; and formulating the motion performance metric based on the constructed biomechanical model.

Once the dedicated application transmits the captured visual data of subject 102 to server 120, the captured visual data is initially processed to identify a number of visual points of interest. The first recognition actions are referred to herein as scene calibration where the real world 20 metre distance is mapped to the interval as captured in the visual data in order to match “image coordinates” to a known real world length. In other words, the known 20 metre interval between distance markers 116 and 118 will be mapped using pixels and horizontal and vertical axes (that is, “x” and “y” axes) such that x/y image coordinates are able to be established so that the real-world length of the captured visual data is known. Where camera 112 is completely stationary (such as one set up on a tripod), scene calibration can be achieved based on any single captured frame of visual data (as the captured visual data and lengths captures will be consistent). For embodiments where camera 112 may not be completely stationary, such as when a person is holding camera 112, scene calibration will be achieved based on normalized image coordinate positions of a plurality of captured frames of visual data. In yet other embodiments where camera 112 may not be completely stationary, scene calibration will be achieved based on normalized image coordinate positions of all of the captured frames of visual data.

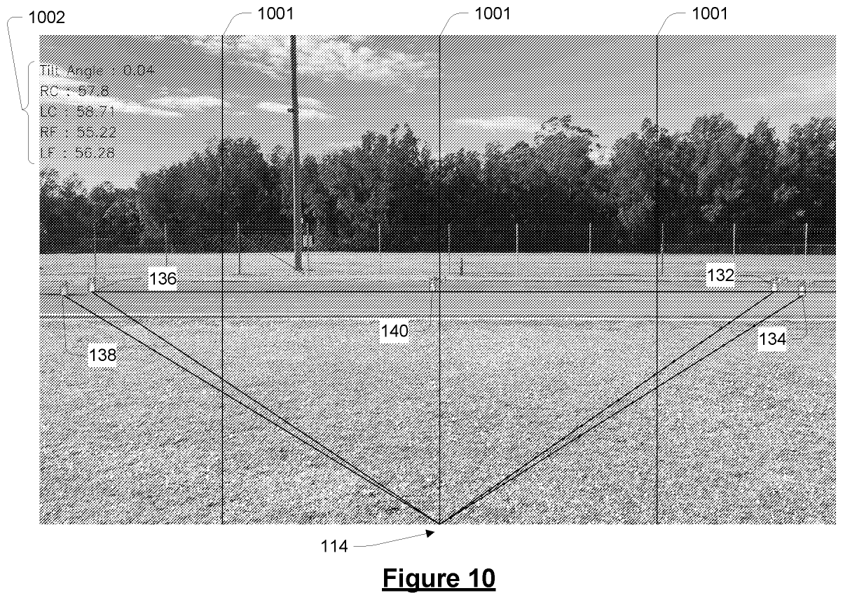

Referring to FIG. 10, a view is shown of the system of FIG. 1, with a number of additional scene calibration annotations. The positioning of marker cones 132, 134, 136 and 138 show is shown as a perspective view such that the angles (compared to a vertical reference lines 1001) of each of the marker cones from camera position 114 are different for each of marker cones 132, 134, 136 and 138. Each of marker cones 132, 134, 136 and 138 and centre marker cone 140 also have a confidence detection value which is a number between 0 and 1 indicating, based on detection techniques described herein, of each cone marker being correctly detected. The maximum confidence detection value is 1 which would indicate with 100% certainty that correct detection has occurred. The minimum confidence detection value is 0 which would indicate that there is 0% chance that correct detection has occurred. In this case, each of marker cones 132, 134, 136, 138 and 140 have respective confidence detection values of 0.85, 0.86, 0.86, 0.8 and 0.79. Further, a panel 1002 is also shown with the following information:
“Tilt Angle: 0.04” refers to a tilt angle (to be discussed further below) which is an angle of which camera 112 is tilted from the horizontal, the tile angle is shown here as 0.04 (or a 4% gradient);
“RC: 57.8” refers to the right side closer cone, in this case marker cone 134, and the angle in respect of vertical reference 1001, the angle is shown here as 57.8 degrees;
“LC: 58.71” refers to the left side closer cone, in this case marker cone 138, and the angle in respect of vertical reference 1001, the angle is shown here as 58.71 degrees;
“RF: 55.22” refers to the right side far cone, in this case marker cone 132, and the angle in respect of vertical reference 1001, the angle is shown here as 55.22 degrees;
“LF: 56.28” refers to the left side far cone, in this case marker cone 136, and the angle in respect of vertical reference 1001, the angle is shown here as 56.28 degrees.

In one embodiment, scene calibration involves comparing the position of marker cones 132 and 134 from the centre of the single captured frame of visual data with respect to marker cones 136 and 138. If, for example, marker cones 136 and 138 are significantly farther from centre than marker cones 132 and 134, a feedback is generated and outputted to the user to move camera 112 closer to marker cones 136 and 138 such that camera position 114 is positioned centrally with respect to distance markers 116 and 118. Additionally, scene calibration involves taking into account the tilt angle of camera 112 with respect to the horizontal. For example, in one embodiment, the tilt angle is determined as an angle between a horizional pixel line and a line joining distance markers 116 and 118 and if the absolute value of the tilt angle is greater than 0.05 (that is a 5% gradient), a feedback is generated and outputted to the user to adjust the tilt of camera 112 accordingly. In yet another embodiment, an IMU sensor value of camera 112 is used to determine to determine camera tilt.

It will be appreciated that in other embodiments, scene calibration is carried out in three dimensions using a horizontal axis, a vertical axis and a depth axis (that is, “x”, “y” and “z” axes) such that x/y/z image coordinates are able to be established.

In other embodiments, the interval between which subject 102 moves is determined after the visual data is captured. For example, subject 102 can be captured moving across an interval of an initially unknown length by the image capture techniques described herein. Once that data is captured, the initially unknown length of that interval is measured and inputted into system 100, for example, by way of the dedicated application. Similarly, in another embodiment, distance markers 116 and 118 are not required and the length of a shoe of subject 102 is used as the real world known length to be inputted into system 100, for example, by way of the dedicated application. In other embodiments, other shoe specifications will be used indirectly to obtain shoe length, for example, a known shoe brand, model and size will have a known length based on manufacturer's specifications. For these embodiments, the same processing techniques described herein are then used to process the captured raw data based on the inputted length.

Further, the captured visual data is processed by server 120 to detect subject 102 as they enter the 20 metre interval from either one of distance markers 116 or 118 (in this case distance marker 116), and track subject 102 moving throughout the 20 metre interval and exiting the 20 metre interval from the other one of distance markers 116 or 118 (in this case distance marker 118). Server 120 is then able to identify human pose points automatically extract kinematic data via pixel mapping using x/y coordinates of each angle of each joint (to be explained in greater detail below) of subject 102 for each frame of video, for example, when a foot of subject 102 connects with the ground point and leaves the ground and providing visualisations of how subject 102 is moving.

As noted above, the motion of subject 102 also enables identification of when a foot of subject 102 touches the ground, referred to herein as ground drop point.

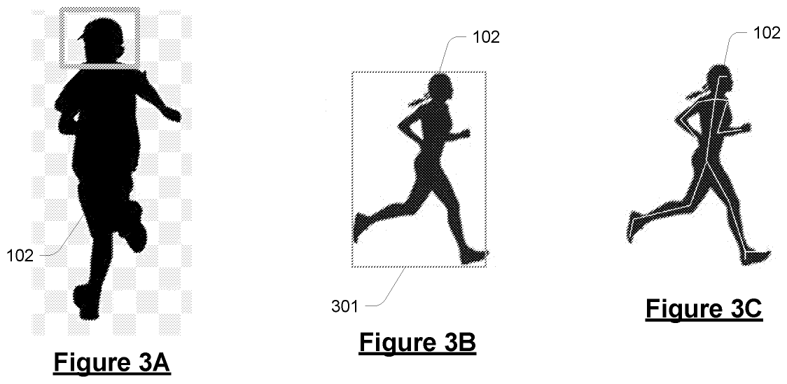

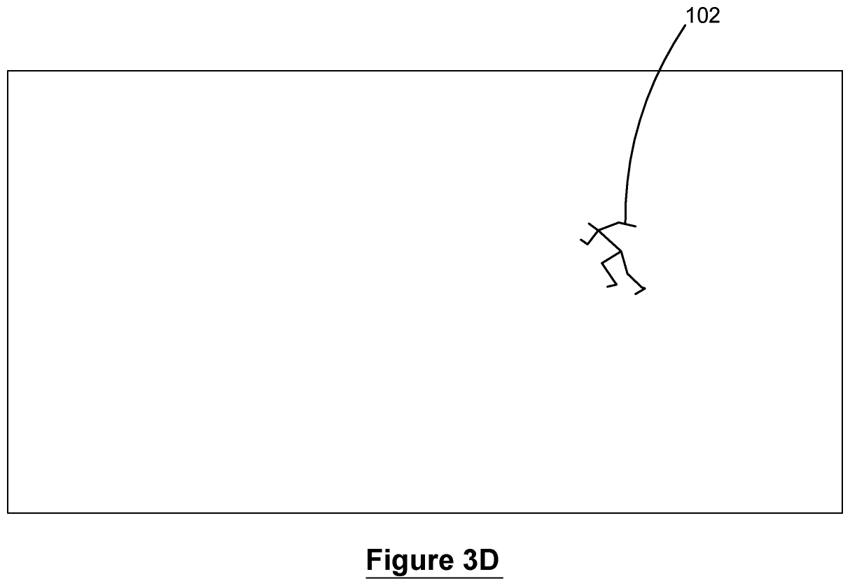

Referring to FIG. 3A subject 102 will be identified using pixel analysis, and in particular through recognition of the face of subject 102. For example, the pixels between the eyes of subject 102 will be used to identify subject 102. From this identification, an athlete profile can be retrieved from the database of server 120 where that athlete has an existing profile or created for an athlete that does not have an existing profile, with the created profile stored in the database of server 120. The existing profile of an athlete may include data and analysis taken from previous visual data captures which can therefore serve as a basis of comparison to the present captured visual data of that same athlete and an appropriate performance metric is formulated (more on this below). Further, athlete profiles will include: athlete date of birth; athlete height and weight at a particular date; past injuries of athlete; and body measurements. It will be appreciated that body measurements are manually inputted into the system and/or gleaned from the captured visual data as will be described further below.

It will be appreciated that system 100 is able to capture the movement of subject 102 in any natural environment, that is not requiring a controlled environment, even if there are other foreign objects including more than one moving human being within the captured visual data. This is achieved in a number of ways, including:
The use of heuristic techniques, for example, selecting from the captured visual data the fastest moving human being in a particular direction (that is, the speed from left to right or right to left) as being as subject 102 (which will be explained further below).
Selecting subject 102 based on the approximate minimum speed requirement such that subject 102 will be identified from the captured visual data based on human being moving at greater than or equal to a specified threshold speed.

It will further be appreciated that through the identification of subject 102, system 100 is able to capture, from a single piece of captured visual data, multiple subjects within the same captured visual data as each subject can be identified, isolated and individually analysed in a similar fashion to a single subject. Once identified and isolated, each of the multiple subjects can be analysed using the techniques described herein in respect of a single subject. However, it will be appreciated that the analysis in respect of each of the multiple subjects by server 120 is carried out in parallel.

Referring to FIG. 3B, once subject 102 is identified, a bounding box 301 will be formed around subject 102 such that bounding box 301 will exactly surround the extremities of subject 102. The extremities of subject 102 will typically be a combination of the feet, knees, elbows, hands and head of subject 102. Referring to FIG. 3C, after bounding box 301 is defined, the human pose points of subject 102 are identified, forming a human pose estimation model that aims to obtain posture of the human body from given human pose points. The human pose points are specific points on a subject that each represent and identify a significant anatomical joint of subject 102. The human pose points shown in FIG. 3C include:
Middle of the head;
Middle of the neck;
Left and right shoulders;
Left and right elbows;
Left and right wrists;
Centre of the hips;
Left and right knees;
Left and right ankles; and
Left and right toes or left and right balls of the feet.

Each of the human pose points will then be able to be tracked using the coordinate axes. The pose estimation model is essentially a line model of each major anatomical component of subject 102, as constructed from the identified human pose points. As seen in FIG. 3C, the major anatomical components of subject 102 are:
The middle of the head to the middle of the neck;
The middle of the neck to the centre of the hips;
The neck to each of the shoulders;
For each arm, the shoulders to the elbows;
For each arm, the elbows to the wrists;
For each leg, the centre of the hips to the knees;
For each leg, the knees to the ankles; and

For each foot, the ankles to the toes.

In one embodiment, the human pose estimation model includes the use of a pre-trained deep learning model on standard dataset (for example, the “Human3.6M” model described http://vision.imar.ro/human3.6m/description.php, incorporated herein by way of cross reference) to train another deep learning model as described in “Toward fast and accurate human pose estimation via soft-gated skip connections”, by Bulat et al, 25 Feb. 2020, (https://arxiv.org/pdf/2002.11098v1.pdf, incorporated herein by way of cross reference). In another embodiment, the human pose estimation model is further fine tuned on dataset created for the underlying use case of subject bio-mechanics analysis. The output of the human pose estimation model is the (x,y) position of a pose point in the image coordinate system as described above and shown in FIG. 3D, termed as ‘estimated human pose model’.

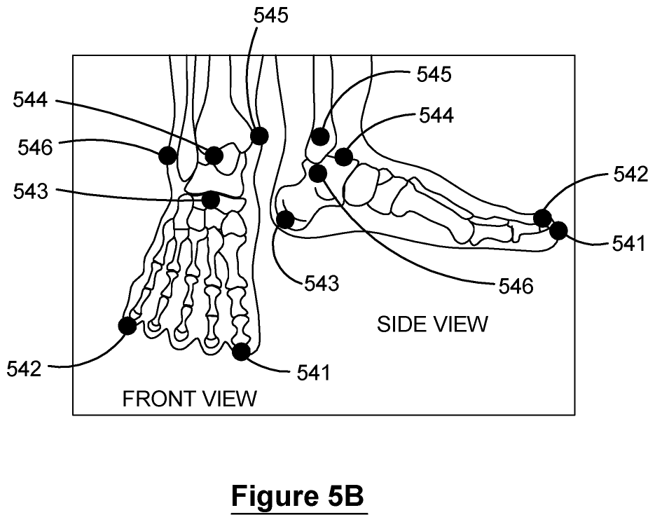

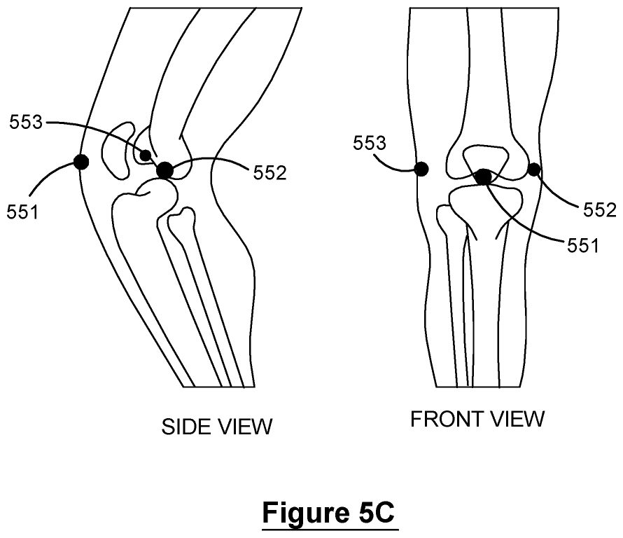

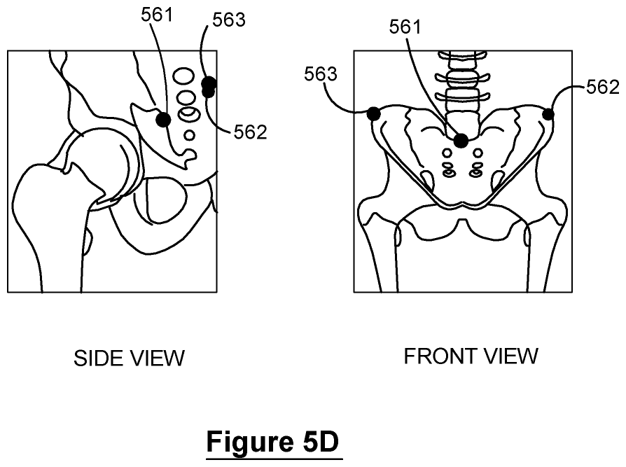

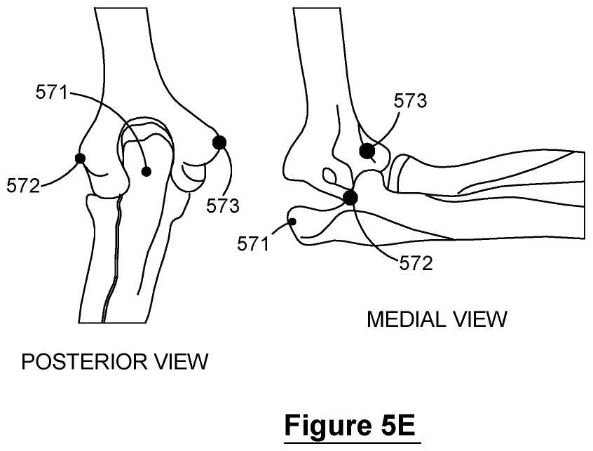

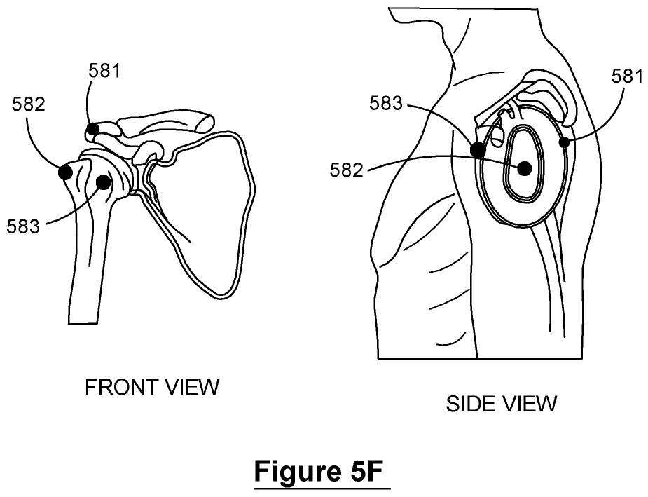

FIG. 5A shows an embodiment with a more detailed pose estimation model comprising 37 mapping human pose points. The 37 human pose points are based on key joints on a human skeleton in order to more closely map human mechanical motion kinematics, the human pose points being on subject 102 at:
Crown, denoted by reference 501;
Eye, denoted by reference 502;
Nostril, denoted by reference 503;
Tragus of ear, denoted by reference 504;
Mouth, denoted by reference 505;
Mid dorsum of wrist, denoted by reference 506;
Lateral epicondyle of elbow, denoted by reference 507;
Contralateral hip flexor, denoted by reference 508;
Patella of front leg, denoted by reference 509;
Midpoint medial knee joint, denoted by reference 510;
Posterior knee joint line of front leg, denoted by reference 511;
Anterior ankle joint line of front foot, denoted by reference 512;
Ankle joint line midpoint medial aspect, denoted by reference 513;
First metatarsal of front foot, denoted by reference 514;
Posterior ankle joint line of front foot, denoted by reference 515;
Heel of front foot, denoted by reference 516;
Occiput, denoted by reference 517;
C7 vertebra, denoted by reference 518;
T2/3 vertebrae, denoted by reference 519;
T4/5 vertebrae, denoted by reference 520;
Midpoint medial elbow joint line, denoted by reference 521;
Midpoint palmer wrist, denoted by reference 522;
L5 vertebra, denoted by reference 523;
Anterior superior iliac spine, denoted by reference 524;
Posterior knee joint line of rear foot, denoted by reference 525;
Ipsilateral knee joint line midpoint, denoted by reference 526;
Patella of rear leg, denoted by reference 527;
Posterior ankle joint line of rear foot, denoted by reference 528;
Heel of rear foot, denoted by reference 529;
Ankle joint line midpoint lateral aspect, denoted by reference 530;
Anterior ankle joint line of rear foot, denoted by reference 531;
Midpoint lateral arch, denoted by reference 532;
First metatarsal of front foot, denoted by reference 533;
Midpoint medial longitudinal arch, denoted by reference 534;
Acromion, denoted by reference 535;
Greater tuberosity, denoted by reference 536; and
Lesser tuberosity, denoted by reference 537.

FIGS. 5B to 5F show the joints of another embodiment of a more detailed pose estimation model in greater detail, with regards to specific positioning of the human points, more specifically:
FIG. 5B illustrates each of the five pose points in each foot, those being at: front of the big toe 541; front of the little toe 542; the heel 543; navicular bone 544; inside of the ankle 545; and outside of the ankle 546.
FIG. 5C illustrates each of the three pose points in each knee, those being at: knee cap or patella 551; left side knee 552; and right side knee 553.
FIG. 5D illustrates each of the three pose points in the hip, those being at: middle hip 561; left hip 562; and right hip 563.
FIG. 5E illustrates each of the three pose points in each elbow, those being at: back ulna 571; right humerus 572; and right humerus 573.
FIG. 5F illustrates each of the three pose points in each shoulder, those being at: acromion 581; greater tuberosity 582; and lesser tuberosity 583.

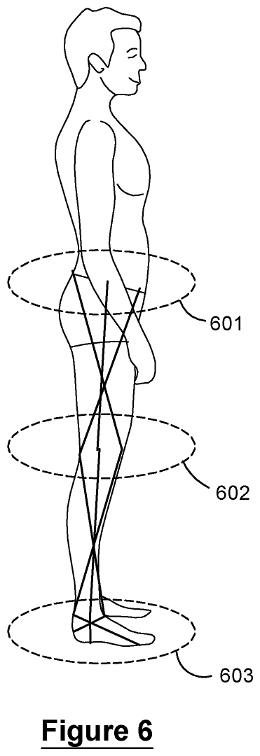

This embodiment utilises a triple point crossover joint system that is best illustrated in FIG. 6. As shown, the legs of subject 102 have triple point joints at the hips denoted by reference 601, the knees denoted by reference 602 and the ankles denoted by reference 603. Each of these triple point joints are joined to the adjacent triple point joint by a diagonal crossover line and centre line between each connection joint, with a similar arrangement for the arms and torso of subject 102. More specifically, the 9 points used from both knees to the hip provides an accurate position of the hip and provides relationship between shoulders and hips. Similarly, the arm positioning is more accurately understood based on the measurement between the three elbow points to the single points at the wrists. Further, the 6 points used for head mapping (those points being at the mouth, nose, eyes, top of the head, back of the head and ears) allows more accurate understanding of the position of the head. If the visual data captured has the ears obscured by hair, the other 5 points are used and will generally be sufficient.

It will be appreciated that in alternate embodiments, any number of mapping points will be utilised. For example, in an embodiment, the three mapping points are used for the legs and arms but not for the rest of the body such as the torso and head/neck. In another example embodiment, the three mapping points are used at the ankles, knees and hips, but only single mapping points are used for the elbows and shoulders. In yet another example embodiment, the head is only a single point. It will be appreciated that various permutations will be used depending on the relevant need for accuracy. For example, a less detailed model may be offered to amateur athlete when such a model will be sufficient, whereas the full 37 point (or more) model may be offered to professional athletes where each and every point must be as accurately measured as possible.

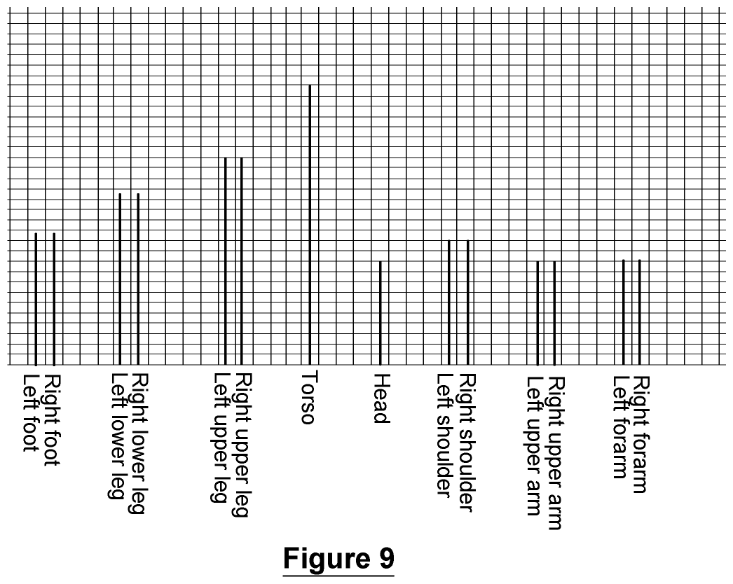

Referring now to FIG. 9, an alternate embodiment of system 100 is shown whereby a limb ratio chart is used to automatically identify the identity of subject 102 in order to retrieve the athlete profile from the database of server 120 where that athlete has an existing profile or created for an athlete that does not have an existing profile, with the created profile stored in the database of server 120. The limb ratios are calculated based on comparisons of length between certain predefined limbs, for example, left foot to right foot, left lower leg to right lower leg, left upper leg to right upper leg, etc. as shown in FIG. 9. The limb ratios are compared to corresponding limb ratios from past captured visual data and if a predefined number of the compared ratios match to a point where a probability threshold is met, the identity of subject 102 is automatically matched to an existing profile (or the retrieved existing profile is presented to the user for confirmation).

In an alternate embodiment of system 100, a combination of movement ratios or patterns are used to automatically identify the identity of subject 102 in order to retrieve the athlete profile from the database of server 120 where that athlete has an existing profile or created for an athlete that does not have an existing profile, with the created profile stored in the database of server 120. The movement ratios are calculated based on comparisons of certain movements, for example, foot rotation, maximum knee height, arm patterns, stride variations and symmetries of stride length or stride frequency, body angles or a combination of different joint angles (for example, maximum knee angle), velocity (in metres per second), and/or ground contact time, amongst others. The movement ratios are compared to corresponding movement ratios from past captured visual data and if a predefined number of the compared ratios match to a point where the probability is high enough that the identity of subject 102 can be matched to an existing profile, subject 102 will be identified as that existing profile (or this will be presented to the user for confirmation).

Further, the shoes worn by subject 102 can also be identified through pixel analysis to specifically identify the shoe brand and model. Based on the specific shoe being used by subject 102, performance of different shoes can then be compared based on parameters including: stride length, stride frequency, ground contact time, airtime, time of run, and top speed, amongst others.

Referring to FIG. 11, system 100 is able to measure the time taken for subject 102 to travel between distance markers 116 and 118. From the captured visual data, two selected human pose points of the torso of subject 102 are identified, those being the hip and the chest of subject 102. The selected human pose points are recognised as passing through a virtual start line at distance marker 116 at a 0 meter point signaling to begin the timer and the selected pose points are subsequently recognised as passing through a virtual finish line at distance marker 118 at a 20 meter point signaling to stop the timer.

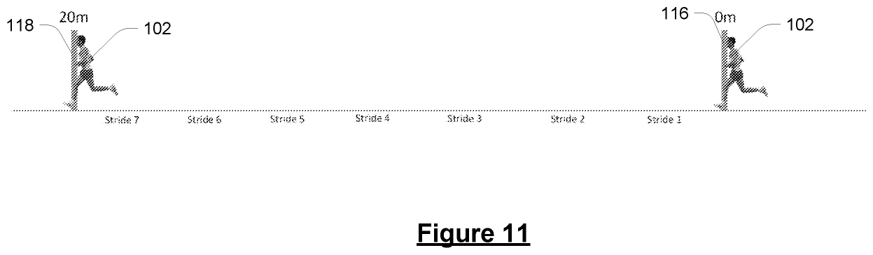

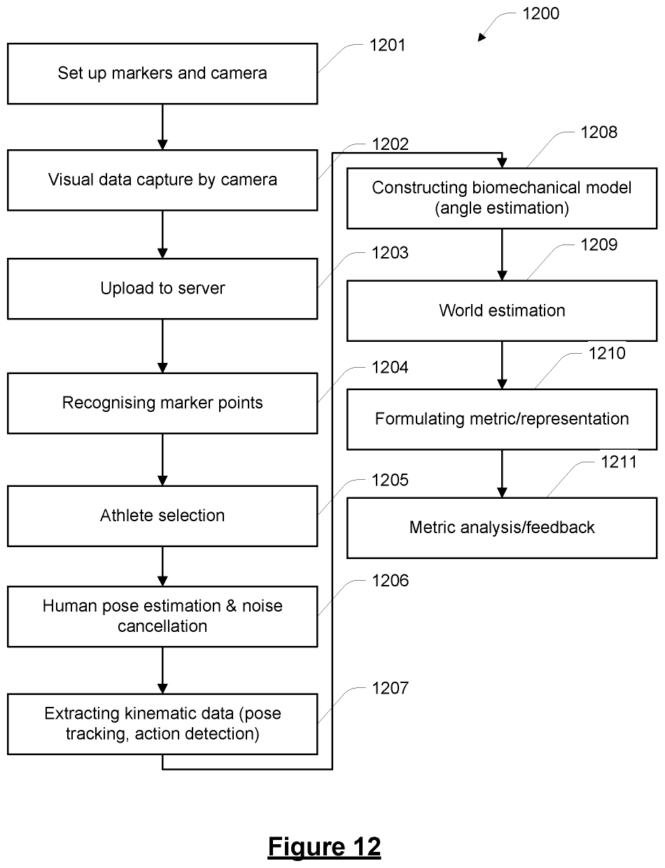

Referring to FIG. 12, there is described a process 1200 for generating a motion performance metric, according to a preferred embodiment. At 1201, each of marker cones 132, 134, 136 and 138 are manually set up on a desired planar surface by a user (of which the user could be subject 102 or another person such as an athlete's coach), as described above, in order to demark the 20 metre interval between distance markers 116 and 118. Marker cone 140 is also placed between distance markers 116 and 118, equidistant (that is, 10 metres from) distance markers 116 and 118. Camera 112 is stationarily mounted on tripod 142 at capture position 114 such that camera 112 is facing marker cones 132, 134, 136, 138 and 140 and that all of marker cones 132, 134, 136, 138 and 140 are within field of vision 113, taking up a significant portion of field of vision 113, and viewable by stationary camera 112 at capture position 114. The user then opens the dedicated application on smartphone 110 so that camera 112 can be used.

At 1202, the user activates camera 112 to record video, that is capture visual data, of the movement of subject 102 from distance marker 116 to distance marker 118. More specifically, prior to subject 102 passing distance marker 116, the recording of the video is activated to commence visual data capture and this continues until subject 102 is beyond distance marker 118, at which point the user deactivates the video recordal to complete the visual data capture. The user will then be prompted by the dedicated application to upload the captured visual data and, at 1203, the user uploads the captured visual data to server 120 where the captured visual data is received for processing.

The movement of subject 102 could be a walk, jog or run, or reverse or sideways movement. Further, the movement could be from a standing start (that is, an acceleration), from a moving start (such as subject 102 running a fly at top speed) or a deceleration.

At 1204, server 120 firstly automatically recognises, from the captured visual data, the distance marker points including distance markers 116 and 118 (to establish the 20 metre interval) and the x/y coordinates as mapped to the real world distance. The detection of distance markers 116 and 118, that is the detection of marker cones 132, 134, 136 and 138, along with centre marker cone 140, is done by way of machine learning techniques to detect each marker cone in an image. In one embodiment, a technique based on deep learning is utilised, as described in “SSD: Single Shot MultiBox Detector”, by Lui et al, 8 Dec. 2015 (revised 19 Dec. 2016), (https://arxiv.org/abs/1512.02325, incorporated herein by way of cross reference). This technique involves “training” server 120 using example images of marker cones captured. In one embodiment, the first frame of the captured visual data is used for detection of marker cones 132, 134, 136 and 138, along with centre marker cone 140. The detected marker cones are identified and annotated with marker bounding boxes that will exactly surround the extremities of each of marker cones 132, 134, 136 and 138, along with centre marker cone 140. In other embodiments, one or more frames are used for detection of marker cones 132, 134, 136 and 138, along with centre marker cone 140. In this case, the results of the multiple frame detections are combined using detection confidence values.

Further, server 120 detects subject 102 using machine learning techniques to detect humans in an image. In one embodiment, the deep learning based technique used for cone detection is utilised. Similarly, this technique involves “training” server 120 using example images of humans. In embodiments where there are multiple humans (one or more may be subjects of interest) present, which is common in a natural environment, tracking of each human is utilised. In one embodiment, the tracking is based on visual and motion similarities between humans detected in two or more consecutive frames, as provided by systems such as those developed by Nano Net Technologies Inc. (https://nanonet.com/blog/object-tracking-deepsort/, incorporated herein by way of cross reference). Such applications of tracking algorithms on human detection results allows the creations of “tracks” which represent distinct unique human subjects (including subject 102) in the captured visual data. In embodiments where subject 102 is the only moving human in the captured visual data, a single track represents subject 102. In embodiments where there is multiple subjects present and detected in the capture visual data, there will be multiple tracks with each track representing one subject. Of these multiple tracks, one or more is selected for further processing by using track characteristics. In one embodiment, track characteristics include the size of the subject in pixels, where the track length in terms of pixels moved during the capture visual data from left to right of the frame is used to select a track as a subject. For example, if the average size of the subject bounding box tracked is greater than 25,000 pixels and the length of the track is greater than or equal to 80% of the width of a frame, the track is a possible subject. In cases where there are two or more tracks which satisfy the above conditions, a further mechanism is used to select a track to be the subject. In an embodiment, this further mechanism includes determining a speed in terms of pixels per frame for each track, and the track which has the fastest speed is selected as a subject track.

As such, server 120 essentially automatically recognises, from the captured visual data, the bounding boxes (of both distance markers 116 and 118 and subject 102) and the plurality of human pose points of subject 102 (to establish both subject 102 and the ground).

As will be appreciated above, any number of human pose points of subject 102 may be identified depending on the requirements of the biomechanical model of that particular embodiment that is to be constructed. From the human pose points, the pose estimation model of subject 102 is formed. At 1205, subject 102 will be identified through the pixel analysis, limb ratio analysis or movement analysis techniques described above and this identification will prompt server 120 to perform a search of its database to check if the recognised athlete has an existing profile and, if so, the associated athlete profile is retrieved from the database. Otherwise, server 120 will communicate to the dedicated application that no existing profile exists that the user will be prompted to create a new profile for the athlete, with the created profile being generated based on information inputted into the dedicated application and stored in the database of server 120.

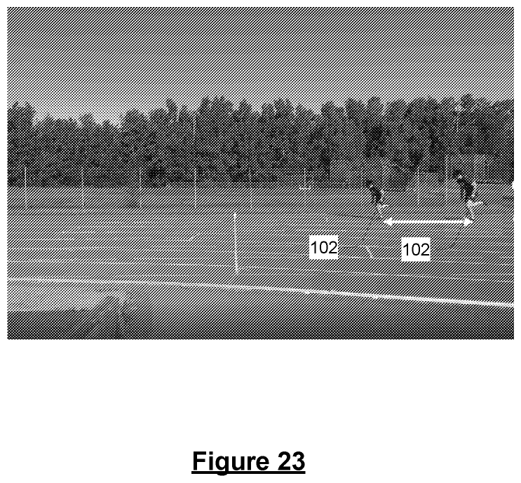

In embodiments where subject 102 is captured moving without the use of distance markers 116 and 118, and shoe length is used to as the real world known length, the detection of subject 102 including human pose points of subject 102 each frame of video when a foot of subject 102 connects with the ground and/or leaves the ground. For example, each consecutive frame where there is a ground touch of subject 102 is identified. FIG. 23 shows the position of subject 102 in consecutive frames, shown in this figure on the same frame for illustrative purposes. Then from the pose estimation model formed from the human pose points, the shoe of subject 102 is detected touching the ground using training of system 100 (similar to the technique using example images for the marker cones) and detecting pose points of the foot (big toe and heel, or the front and end of the foot). For a ground touch frame, the detected pose points of big toe and heel are allocated x/y pixel coordinates, for example, the toe is x1, y1 and the heel is x2, y2, and the known shoe length (in centimetres, in this embodiment) will equal the pixel length between x1 and x2, thus being able to calculate pixels per centimetre. In preferred embodiments, the process of calculating pixels per centimetre is carried out for two or more of the consecutive frames where there is a ground touch of subject 102, with each calculated pixels per centimetre averaged.

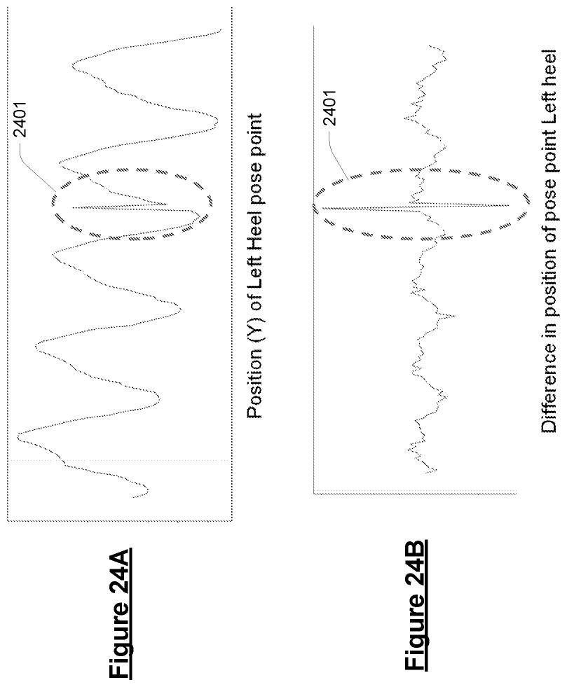

At 1206, the human pose points will be identified on subject 102 from the visual data and it will undertake a noise cancellation process whereby subject 102 can be isolated within the 20 metre interval, and which will include appropriate cropping and clipping of the visual data. It will be appreciated that the human pose estimation model may encounter errors in estimating human pose points. A pose data correction module is provided herein aims to correct any errors in pose data points. In an embodiment, the “smooth” change in position of a pose point across frames is assumed to identify noisy pose points that are indicative of a pose point detection error. Referring to FIG. 24A, there is shown an example of y-coordinate position plot over time for a left heel point of subject 102 with a noisy pose point 2401 shown which is clearly identifiable. To identify noisy data for a particular pose point, for example the left heel, the difference in consecutive data values for two frames is calculated, which is shown as plotted over time in FIG. 24B. The difference value is then used to estimate a gaussian mean and variance parameters. A difference value which is has a variance of more than 2 times from the mean is classified as a noisy data point. Any noisy data point is replaced by mean of pose points in two neighbouring frames (the frame prior and the frame following the frame with the erroneous pose point).

At 1207, server 120 extracts kinematic data of subject 102 based on the pose estimation model of subject 102 along with scene calibration information. Such kinematic data includes the relative position of human pose points in respect of the ground and/or each other and angles between major anatomical components of subject 102, amongst others. The pose tracking (and noise cancellation, that will enable more efficient detection) will then enable the specifics of the movement of subject 102 for example, specific stride related information, to be gleaned. Further, where multiple athletes are captured (and therefore more than a single subject 102), each of these subjects can be split from the captured visual data and isolated for individual analysis.

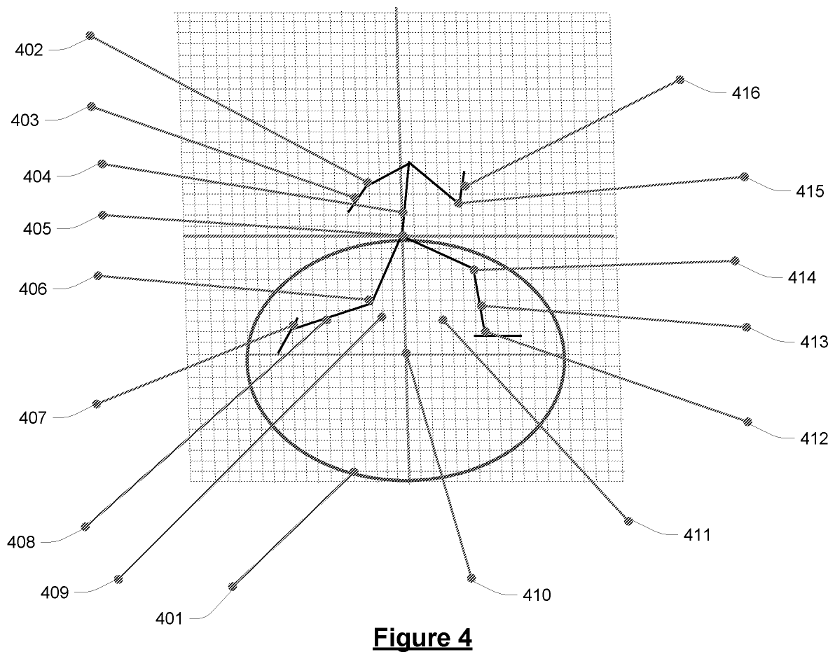

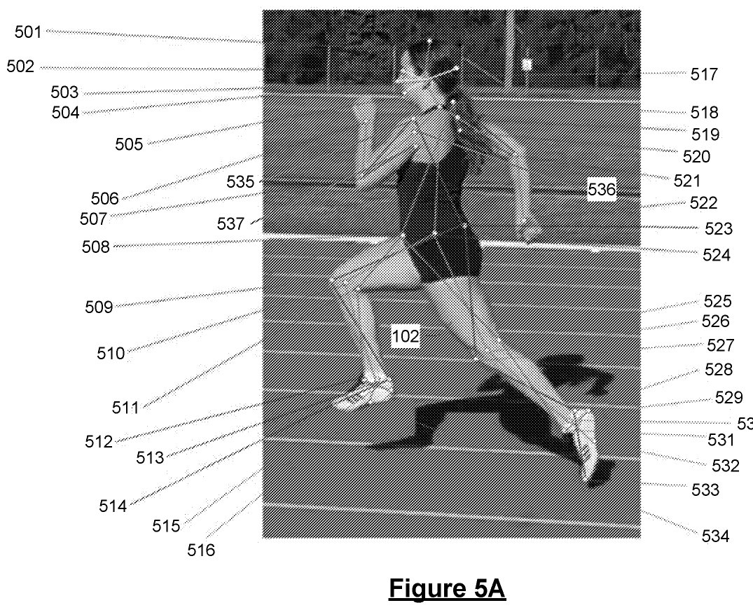

At 1208, server 120 uses kinematic data of subject 102 to form the biomechanical motion model of subject 102. This includes pose position tracking of subject 102 over the 20 metre interval such that the various different poses of subject 102 are recognised at predetermined points within the 20 metre interval that are compared through the biomechanical model. Referring to FIG. 4, the biomechanical model includes the position of pose points and requires normalisation of the hip position of subject 102. Hip normalisation refers to using a central pose point, such as the pose point of the centre of the hips, as a central reference point 401 of which the body movement of subject 102 is examined relative to central reference point 401 in order to identify changes in physical motion shape of subject 102. This biomechanical model is visually displayed using a grid coordinate map with a central vertical axis 402 perpendicularly intersecting a central horizontal axis 403 at the central reference point 401 and the central vertical axis 402 perpendicularly intersecting a lower horizontal axis 404 at a lower reference point 405. It will be appreciated that in other embodiments, coordinate types other than a grid are used, for example concentric circles about the central reference point and radial lines outwardly extending from the central reference point.

At 1209, the environment of subject 102 (referred to as “World estimation”) can be gleaned from both distance markers 116 and 118 and the plurality of human pose points on subject 102 to map the captured visual data to the real-world environment. In particular, ground drop point is established and with this the specific impact points with the ground for subject 102 in motion, which identifies where the strides are taken to begin and end.

At 1210, a performance metric is formed. The performance metric is any qualitative based assessment or result of the analysis of the movement of subject 102 including the formed biomechanical model. Performance metrics include speed, velocity, acceleration, angular speed of joints, angles and change in angles during motion, stride frequency, stride length, ground contact time, air time, and other temporal-based performance metrics, amongst others. The performance metric can take a number of forms including but not limited to: numbers or scores, overlayed visual information on a video, graphs, and other dynamic representations that adjust over time such as a new video being created isolating subject 102, or a dial type graphic. A number of examples are illustrated in the Figures and will each be described below. Finally, at 1211, based on the formed performance metric or representation, feedback is visually displayed by smartphone 110, a number of examples of which will also be described below.

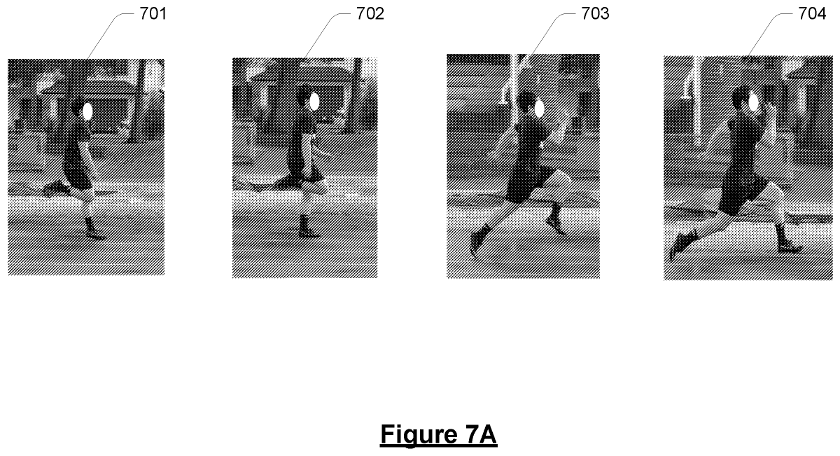

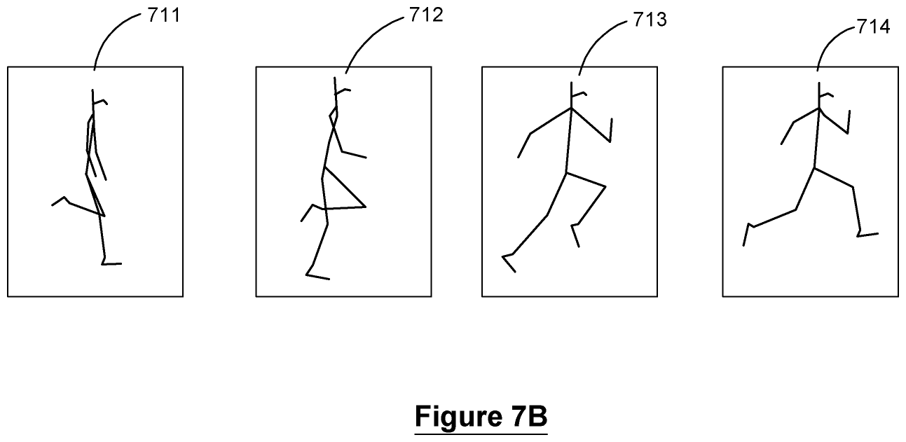

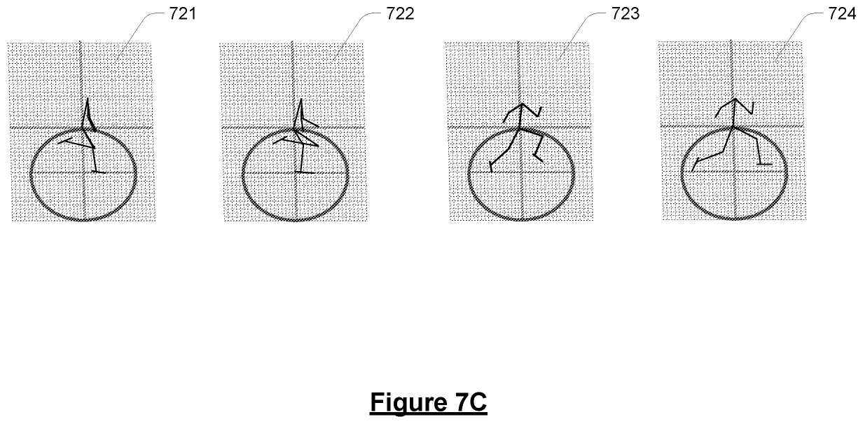

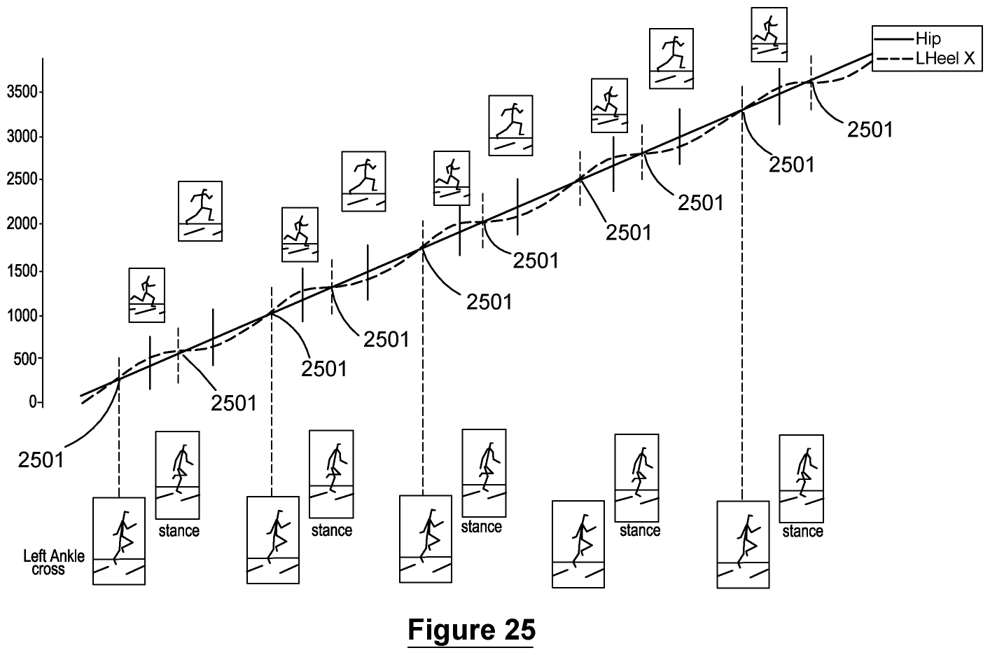

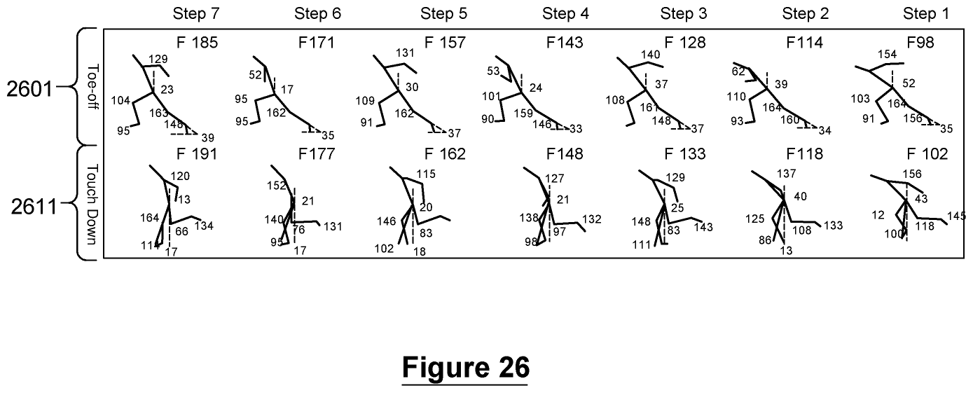

Referring to FIGS. 7A to 7C, there is illustrated a specific biomechanical model that automatically identifies the precise frames of the captured visual data where subject lifts their back foot off the ground to push off on a stride, referred to herein as a “toe-off” biomechanical model. This model uses the very last frame before the back toe of subject 102 leaves the ground, and will therefore represent angles, shapes and position of subject 102 at a “toe-off” or “foot up” point (where the back foot it just about to leave the ground). Additionally, a similarly constructed biomechanical model could be based around the other major key temporal phase frames of a gait cycle, those being a “touch down” or “foot down” point (where the front foot, either left or right, is just about to make contact with the ground) and a full support or mid stance phase (where the foot flattens on the ground and when hip and heel points are vertically aligned). In one embodiment, system 100 uses images of a specific action and trains an image classifier for the three stance classes (foot up, foot down and mid stance) by way of a deep learning based image classification method, as provided by open-source algorithm platform OpenMMLab (https://github.open-mmlab/mmclassification, incorporated herein by way of cross reference). In another embodiment, two different classifiers are trained using natural images (such as raw photo or video stills) and stick figure images (such as human pose estimation model outputs). The overall classification results are a weighted sum of the output of the two classifiers. In an embodiment, equal weights are given to the two classifiers, but in other embodiments, differing weights are applied. Referring to FIG. 25, in yet another embodiment, system 100 uses pose points (x/y coordinate) position data as a time series signal. In this case a combination of pose points for hip ‘x’ position and left heel ‘x’ position are plotted linearly over time and where the lines intersect will denote intersection frames, denoted by reference 2501.

Looking more closely at the detection of the three stance classes (foot up, foot down and mid stance), this can be separated into the following steps: (i) Training Step; and (ii) Testing/Inference Step. Training step (i) includes:

A. Regressor: where a regressor is used to estimate a curve for movement from ground drop point data. Specifically, two curves each for foot up and foot down are learned, followed by a curve correlating the movements of the left and right foot of subject 102. The individual curves depict the individual frequency of left and right foot up and foot down of subject 102 along with continuity as subject 102 is tracked. The correlation curve represents the frequency interval between consecutive foot up and foot down frames. The regression parameters include frame rates and pose points for each of the left and right feet of subject 102.

B. Visual Analysis: where, based on the human pose points, a trajectory of movement is fitted on the region between the marker cones on respective frames. The features from an “N×N” area are extracted within frames of capture visual data and a binary classifier is trained. The feature extraction selectively enhances the frame by a factor of two around the pose points using a deep neural encoder-decoder network. The network takes a low resolution patch as input, and then outputs a high resolution patch. The shoe of subject 102 is then detected in the frame using a shoe detector neural network, the shoe detector neural network being conditioned on the pose points. Based on the detection, a K dimensional feature vector is extracted and this feature vector is used to train the binary classifier, where K represents a number choice which controls the complexity of the model. In one embodiment K is set at 512. The classifier provides “foot touch”/“foot not touch” (equivalent to “foot down”/“foot up”) as labels along with the confidence of a correct detection.

C. Once the shoe of subject 102 is detected, a bounding box is formed around the shoe and this box is segmented to obtain subpixel level points of the shoe tip. A density detection mechanism is used to then detect the toe of the shoe and scale-invariant feature transform (SIFT) features are extracted from the image frame and labelled as lying towards ground or not (a classifier is trained based on such labelling). The input to the classifier are SIFT keypoints at the subpixel level. Then, Density-Based Spatial Clustering of Applications (DBSCAN) to is used to identify the cluster with similar classification. When testing, clustering along with ground plane estimation is used to filter whether or not the foot of subject 102 is touching the ground.

Testing/Inference Step (ii) includes:

A. When testing commences, the initial human pose points are identified and confidence of detection of those pose points is determined by fitting the pose points on the individual regression curves and the correlation curve. If an anomaly is identified, a correction based on the learned curves is triggered. If the required correction is spatial and within a pre-identified spatial radius of the detected pose point, the pose point is corrected.

B. However, if the correction is beyond the threshold or the number of required corrections is high (for example, a certain percentage of the number of “foot up” frames and “foot down” frames), a visio-temporal post processing is triggered and the detected pose points are classified for each frame. If the classification from the visio-temporal processing detects that the foot of subject 102 was not touching the ground in the current frame (for a “foot down”), a temporal analysis is triggered within the a window of <t> frames from the detected frames, with the window ‘t’ being obtained from the frequency interval of the regression curves. The “foot up” and “foot down” is then detected at subpixel level using techniques outlined above at (i).

In another embodiment, a combination of the above described detection methods is used and the results are combined using a weighted combination of results from image classification.

Referring to FIGS. 7A to 7C, there is illustrated the four phases of interest for a running motion, those being the three phases or stance classes noted above (touch down, mid stance and toe-off) along with the fourth phase of “mid flight” which is classified as the pose whereby there is the furthest distance or split between the front and back thigh of subject 102. It will be appreciated that the “mid flight” phase is detected in the same fashion as the other three stance classes which is set out above. Each of the phases is also known as an action event. More specifically FIG. 7A illustrates four video frames where subject 102 is in the touch down, mid stance, toe-off and mid flight phases respectively denoted by references 701, 702, 703 and 704. Referring to FIG. 7B, there is illustrated four estimated human pose models or phases where subject 102 is in the touch down, mid stance, toe-off and mid flight phases respectively denoted by references 711, 712, 713 and 714. A human pose estimation model is applied to subject 102 moving across the 20 metre interval and server 120 automatically identifies each of the four phases through the above described stance detection processes. The extracted line model images undergo the hip normalisation process by being resized, which is best shown in FIG. 4 which shows a more detailed version of 724, so the hip of subject 102 aligns with the central reference point, and the foot aligns with the lower reference point representing the ground with the central vertical axis perpendicularly intersecting the central horizontal axis at the central reference point the and the central vertical axis perpendicularly intersecting the lower horizontal axis at the lower reference point. FIG. 7C illustrates where each pose estimation model of FIG. 7B is overlayed onto a grid coordinate map having a centre cross reference and a lower cross reference where each of the touch down, mid stance, toe-off and mid flight phases to create respective biomechanical models each phase denoted by references 721, 722, 723 and 724 respectively. Similar to FIG. 4, the biomechanical models of FIG. 7C are visually displayed using a grid coordinate map representing the geographical positioning of the body of subject 102. Referring again to FIG. 4, a circle reference 401 is used to identify positioning of front side and back side mechanics of subject 102. FIG. 4 also shows the following features of biomechanical model 724:
At reference 402, the angle and position of rear elbow against hip position, centre line and grid reference.
At reference 403, there indicates rear forearm angle, position and distance from the hip and neck.
At reference 404, there indicates torso angle from hip to neck against centre line and grid reference.
At reference 405, the entre line for hip alignment (central reference point).
At reference 406, the angle and position of rear knee against hip position centre line and grid reference.
At reference 407, the plantar flexion and angle position of rear ankle and foot on ground and against centre line, hip position and grid reference.
At reference 408, there indicates horizontal and vertical rear shin angle and position.
At reference 409, the top left segment indicates rear side biomechanical model.
At reference 410, there indicates ground reference (lower reference point).
At reference 411, the top right segment indicates front side biomechanical model.
At reference 412, the dorsi flexion flexion angle position of front ankle and foot in air and against centre line, hip position and grid reference.
At reference 413, there indicates horizontal and vertical front shin angle and position.
At reference 414, the angle and position of front knee against hip position centre line and grid reference.
At reference 415, the angle and position of front elbow against hip position, centre line and grid reference.
At reference 416, there indicates front forearm angle, position and distance from the hip and neck.

In other embodiments, in addition to circle reference 401, an upper body circle reference is also used to identify positioning of front side and back side upper body mechanics of subject 102.

As shown in FIGS. 4 and 7C, vertical and horizontal cross references are established through the hip of subject 102. This allows the identification of the following positions of subject 102:
A. Lean of the torso which is measured from the hip to the centre of the neck.
B. Position of front foot from centre line.
C. Knee height off the ground.
D. Level of dorsi-flexion of the front foot (ankle angle between shin and foot).
E. Position of front and rear arm in relation to opposite legs.

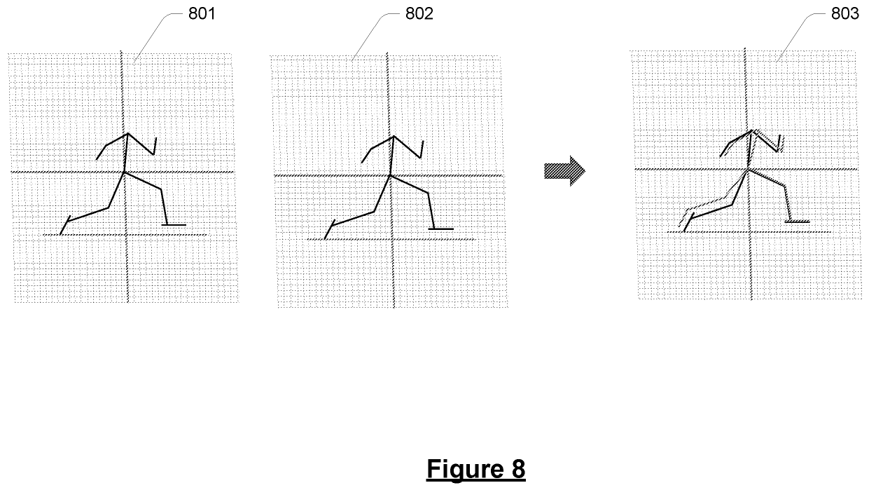

The model is used to provide a performance metric represented by different instances overlayed on each other, as shown in FIG. 8, whereby the same toe-off pose of subject 102 taken at different times (in this case two different times denoted by references 801 and 802) is directly overlayed to identify changes of biomechanics at that position, the overlay denoted by reference 803. This model will also allow the comparison of an athletes faster runs to provide feedback on what shapes and angles have resulted in the athlete performing at their best. Further, the model can identify how an athlete runs differently (in terms of position) on different surfaces such as grass or tartan racetrack. Yet further, the model can show the difference in mechanics for a run over wickets as compared to a run over no wickets.

In other embodiments, the biomechanical model is based on other key frames and the subject position, such as a highest knee lift point and corresponding knee angles.

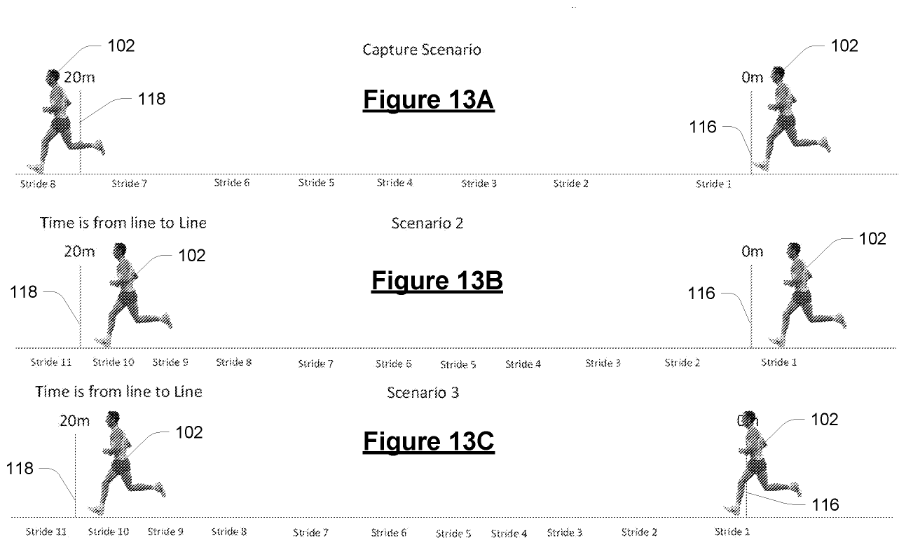

Referring to FIGS. 11 and 13A to 13C, an embodiment of a displayed performance metric takes the form of a to scale horizontal 2D representation of 20 metre interval showing distance markers 116 and 118 and showing the stride points of subject 102. It is noted that the stride points are the points along the ground where the feet of subject 102 impact the ground and the patterns of strides will provide certain information on the movement of subject 102. Referring specifically to FIG. 11, a scenario is captured whereby subject 102 has 7 stride points within the 20 metre interval with relatively even strides being recorded potentially indicating a consistent speed of movement. Referring specifically to FIG. 13A, a scenario is captured whereby subject 102 has 7 stride points within the 20 metre interval, also showing an 8th stride point beyond distance marker 118. The spacing of the stride points is initially relatively wide, then for the 3^{rd} to 6^{th} stride points, the strides are smaller in length before widening again. This pattern potentially indicates a change of speed, for example a deceleration of subject 102 followed by an acceleration. Referring specifically to FIG. 13B, a scenario is captured whereby subject 102 has 9 stride points within the 20 metre interval, also showing a 1^{st} stride point prior to distance marker 116 an 11^{th} stride point beyond distance marker 118. The spacing of the stride points is relatively small potentially indicating a change in stride frequency compared to the scenario of FIG. 13A. Alternatively, this may also potentially indicate a slower pace (for example a walk, rather than a run) being recorded. Referring specifically to FIG. 13C, a scenario is captured whereby subject 102 has 10 stride points within the 20 metre interval, also showing an 11th stride point beyond distance marker 118. The spacing of the stride points is initially relatively wide, then for the 3^{rd} to 6^{th} stride points, the strides again are even smaller in length than those shown in FIG. 13B.

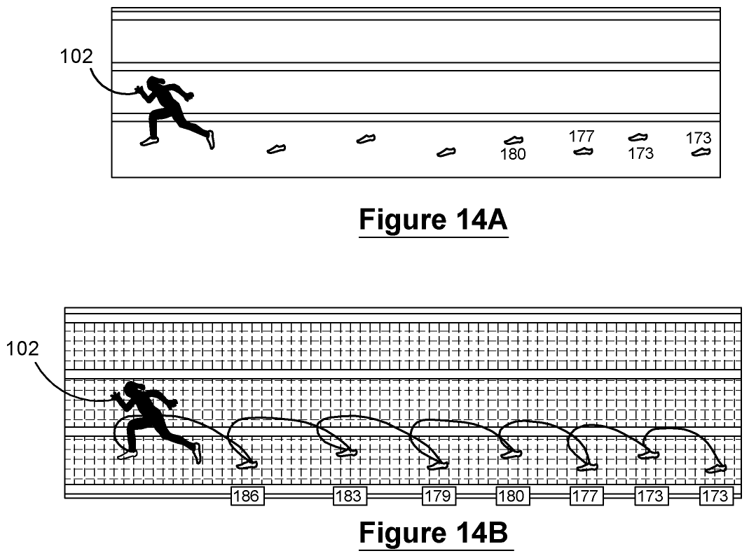

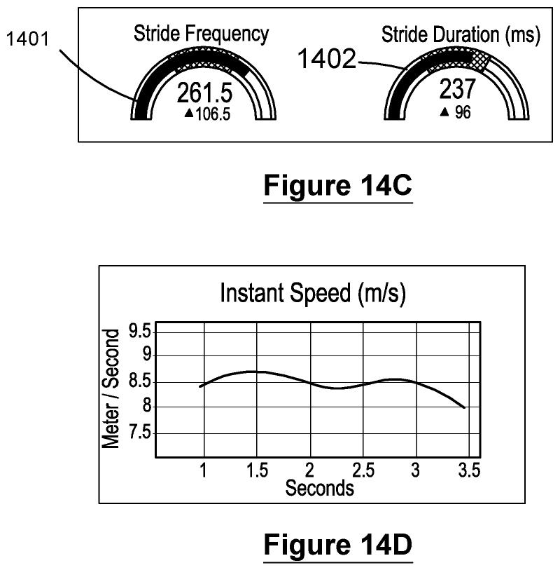

Referring to FIGS. 14A to 14D, further embodiments of a displayed performance metric/representation take the form of to scale horizontal 2D representations of the 20 metre interval showing the stride points of subject 102 on a customised background with a particular focus on the positioning of the feet of subject 102 and associated stride frequency and duration. Referring specifically to FIG. 14A, there is illustrated an embodiment showing the 20 metre interval including stride points shown as shoe icons and labelled with the stride length (the examples shown, in the order indicated by the silhouette representation of subject 102, being 1.73m, 1.73m, 1.77m and 1.80m). It will be appreciated that subject 102 may have actually been captured in one setting, such as a public park, but system 100 can place subject 102 at another setting such as a stadium with a racetrack. In other words, system 100 is able to isolate subject 102 from the captured visual data and superimpose subject 102 into any setting. As shown in FIG. 14C, there is displayed two dynamic dials, denoted by references 1401 and 1402, respectively showing the stride frequency (in strides per second or strides per minute) and stride duration (in milliseconds) of subject 102 as subject 102 moves within the 20 metre interval. It will be appreciated that the readings will change throughout the movement of subject 102 and that those displayed are captured at a certain point in the movement of subject 102. Each dynamic dial 1401 and 1402 also has a dynamically changing number of the present stride frequency and stride duration which again, will change throughout the movement of subject 102 but are shown as captured at a certain frame of the captured visual data. Referring specifically to FIG. 14B, there is illustrated an embodiment showing the 20 metre interval including stride points shown as shoe icons and showing stride trajectory of a selected marker point (in this case the right foot) of subject 102. It will be appreciated that multiple key human pose points of subject 102 can be selected and displayed on the same or different representations of 20 metre interval. As shown in FIG. 14D, there is displayed a performance metric taking the form of a line graph showing the “instant” speed in metres per second of an athlete over the course of a few seconds, in this case starting at a time point just prior to 1 second and going up to about 3.5 seconds.

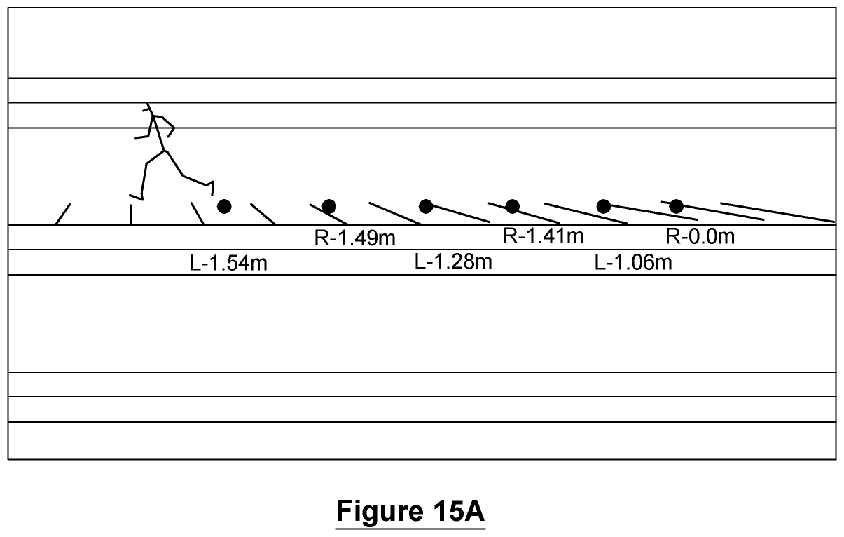

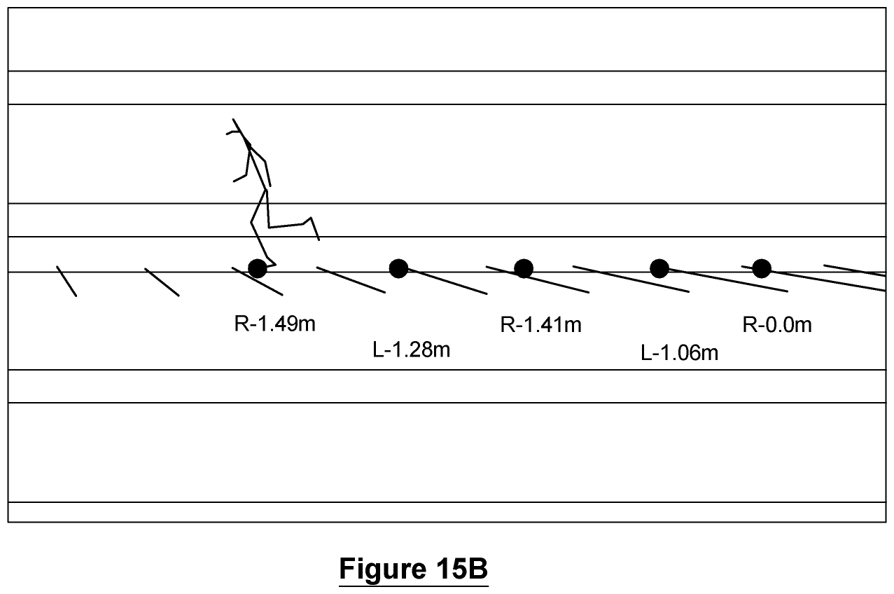

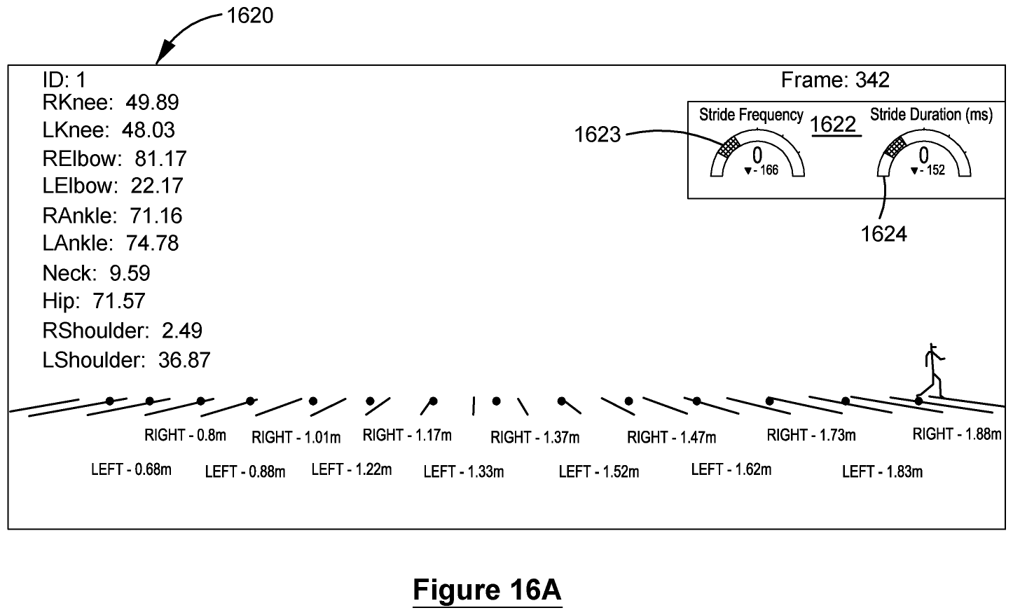

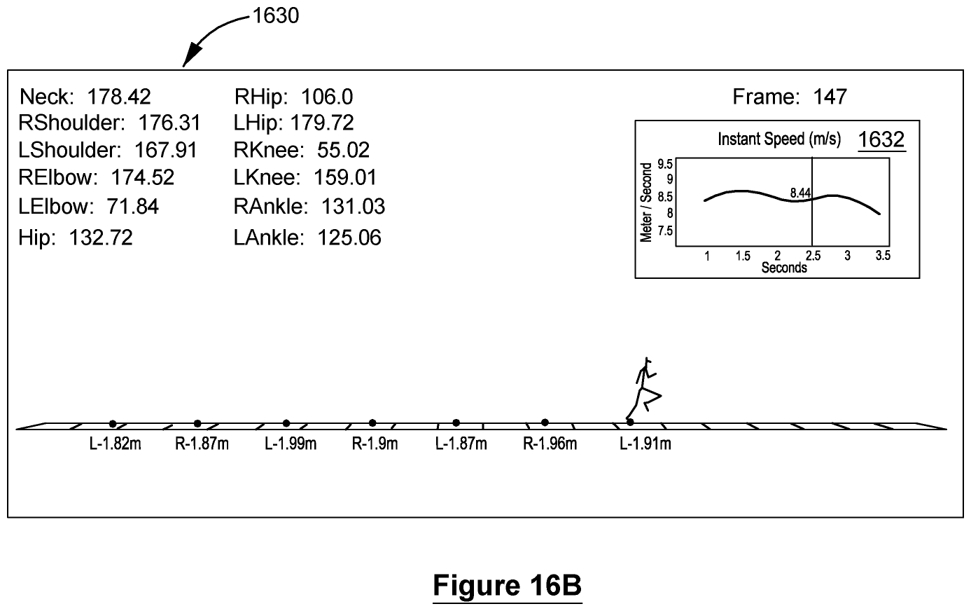

Referring to FIGS. 15A, 15B, 16A and 16B, there is shown alternate embodiments of a displayed performance metric/representation take the form of to scale horizontal 2D representations of at least a portion of the 20 metre interval showing the stride points of subject 102. Referring specifically to FIG. 15A, there is illustrated an alternate embodiment showing a portion of the 20 metre interval where subject 102 is in a completely airborne pose (not touching the ground, “toe-off” or “foot up”) and the stride points are labelled with a metre reading of the stride length for each of the left and right feet, the examples showing a first right stride point having a length reading of 0.0m (given it is the first step, there is no reading), two further stride points for right stride having respective lengths of 1.41m and 1.49m and three stride points for a left stride having respective lengths of 1.06m, 1.28m and 1.54m. Referring specifically to FIG. 15B, there is illustrated an alternate embodiment showing a portion of the 20 metre interval where subject 102 has one foot on the ground (“touch down” or “foot down”) and the stride points are labelled with a metre reading of the stride length for each of the left and right feet, the examples showing a first right stride point having a length reading of 0.0m (given it is the first step, there is no reading), two stride points for right stride length of 1.41m and 1.49m and two stride points for left stride length of 1.06m and 1.28m. Referring specifically to FIG. 16A, there is illustrated an alternate embodiment showing the stride points of subject 102 within the 20 metre interval. Each stride point is labelled with a metre reading of the stride length for each of the left and right feet, the examples showing seven stride points for left stride lengths of 0.68m, 0.88m, 1.22m, 1.33m, 1.52m, 1.62m, and 1.83m and seven stride points for right stride lengths of 0.8m, 1.01m, 1.17m, 1.37m, 1.47m, 1.73m, and 1.88m. Also shown at 1620 is a list displaying the angles in degrees for a plurality of human pose points at specific joints of subject 102, those being: right knee joint (“RKnee”) at an angle of 49.89 degrees; left knee joint (“LKnee”) at an angle of 48.03 degrees; right elbow joint (“RElbow”) at an angle of 81.17 degrees; left elbow joint (“LElbow”) at an angle of 22.17 degrees; right ankle joint (“RAnkle”) at an angle of 71.16 degrees; left ankle joint (“LAnkle”) at an angle of 74.78 degrees; neck (“Neck”) at an angle of 9.59 degrees from the vertical; hip joint (“Hip”) at an angle of 71.57 degrees from the horizontal; right shoulder joint (“RShoulder”) at an angle of 2.49 degrees; and left shoulder joint (“LShoulder”) at an angle of 36.87 degrees. These joint angle metrics will dynamically change as subject 102 moves between the 20 metre interval. There is also displayed a video frame number, in this case “Frame: 342”, showing the precise frame of video corresponding to the position of subject 102. This will enable certain frames of interest showing subject 102 is a particular pose, to be identified. At 1622, there is displayed two dynamic dials, denoted by references 1623 and 1624, respectively showing the stride frequency (in strides per second or strides per minute) and stride duration (in milliseconds) of subject 102 as subject 102 moves within the 20 metre interval. It will be appreciated that the readings will change throughout the movement of subject 102 and that those displayed are captured at a certain point in the movement of subject 102 (that being at “Frame: 342” noted above). Each dynamic dial 1623 and 1624 also has a dynamically changing number of the present stride frequency and stride duration which again, will change throughout the movement of subject 102 but are shown as captured at “Frame: 342”. Referring specifically to FIG. 16B, there is illustrated an alternate embodiment showing the stride points of subject 102 within the 20 metre interval. Each stride point is labelled with a metre reading of the stride length for each of the left and right feet, the examples showing four stride points for left stride lengths of 1.82m, 1.99, 1.87, and 1.91 and three stride points for right stride lengths of 1.87m, 1.9, and 1.96. Also shown at 1630 is a list displaying the angles in degrees for a plurality of human pose points at specific joints of subject 102, those being: the neck with respect to the torso (“Neck”) at an angle of 178.42 degrees; right shoulder joint (“RShoulder”) at an angle of 176.31 degrees; left shoulder joint (“LShoulder”) at an angle of 167.91 degrees; right elbow joint (“RElbow”) at an angle of 174.52 degrees; left elbow joint (“LElbow”) at an angle of 71.84 degrees; hip joint (“Hip”) at an angle of 132.27 degrees from the torso; right side hip joint (“RHip”) at an angle of 106.00 degrees from the right leg; left hip joint (“LHip”) at an angle of 179.72 degrees from the left leg; right knee joint (“RKnee”) at an angle of 55.02 degrees; left knee joint (“LKnee”) at an angle of 159.01 degrees; right ankle joint (“RAnkle”) at an angle of 131.03 degrees; and left ankle joint (“LAnkle”) at an angle of 125.06 degrees. These joint angle metrics will dynamically change as subject 102 moves between the 20 metre interval. There is also displayed a video frame number, in this case “Frame: 147”, showing the precise frame of video corresponding to the position of subject 102. This will enable certain frames of interest showing subject 102 is a particular pose, to be identified. At 1632, there is displayed a dynamic “instant” speed line graph similar to that illustrated in FIG. 14C. It will be appreciated that the readings will change throughout the movement of subject 102 and that those displayed are captured at a certain point in the movement of subject 102 (that being at “Frame: 147” noted above). Further, dynamic graph 1632 will change throughout the movement of subject 102 but is shown as captured at “Frame: 147”.

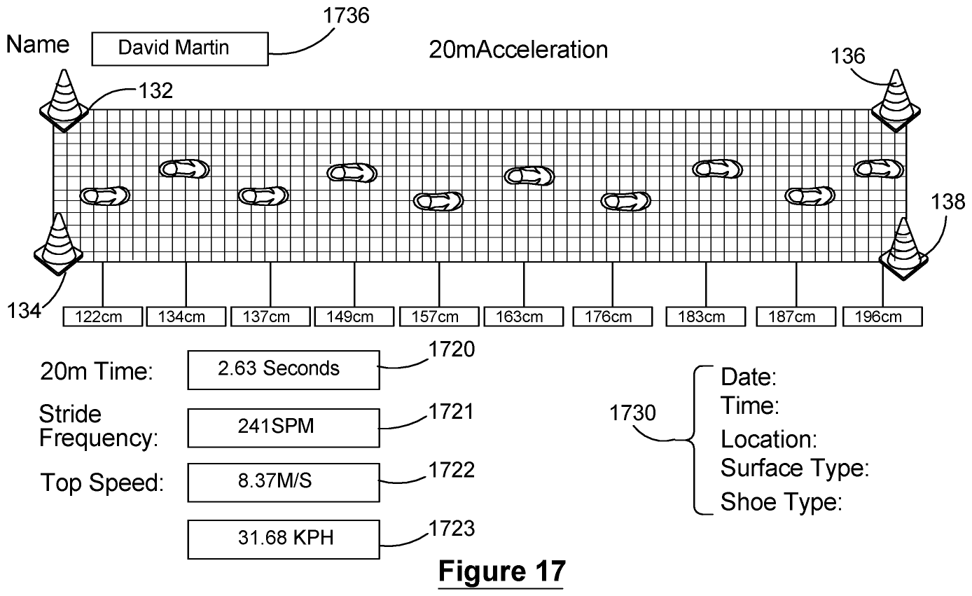

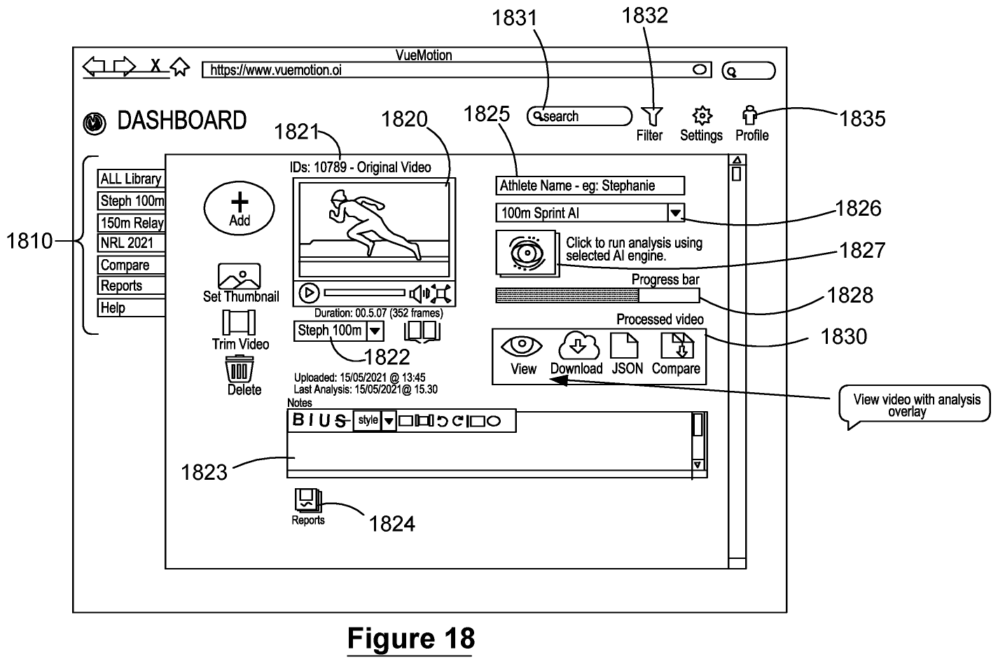

Referring to FIG. 17, there is shown an embodiment of a displayed performance metric in the form of a to scale top view horizontal 2D representation of the 20 metre interval. In this embodiment, marker cones 132, 134, 136 and 138 are displayed along with the stride points of subject 102, with each stride point labelled with the stride length (the examples shown being, in order, 122 cm, 134 cm, 137 cm, 149 cm, 157 cm, 163 cm, 176 cm, 183 cm, 187 cm, and 196 cm). This embodiment shows subject 102 accelerating over the 20m interval. Further, some numerical metrics are also providing the form of: the time the subject takes across the 20m interval 1720; the stride frequency across the 20m interval 1721; and the top speed of subject 102 in both metres per second 1722 and kilometres per hour 1723. Other information is provided at 1730, including: date of the visual data capture; time of visual data capture (which could be either the start time or end time); location of the visual data capture; surface type (racetrack, asphalt, grass etc.); and shoe type (including model and make). Further, a name of the athlete is displayed at 1735 (shown as “David Martin”) and a title is also displayed (shown as “20m Acceleration”).

Referring to FIG. 18, an embodiment of a webpage containing multiple graphical elements that comprise the webpage. Such a display is viewable as part of the dedicated application on smartphone 110 or within a web browser application. In the illustrated example, a dashboard view is shown with a number of tabs 1810 (in this example, labelled: “All Library”; “Steph 100m”; “150m Relay”; “NRL 2021”; “Compare”; “Reports”; and “Help”) with the “All Library” tab at the foreground. Tabs 1810 denote different user created folders for managing videos based on events or specific dates. There is also displayed a video display field 1820 with each video having an unique associated identification number 1821, “ID #”, that is displayed above video display field 1820. The video will also have a name 1822 and a notes section 1823. Further, reports 1824 will be quickly accessible through links on the dashboard, as well as via the relevant tab of tabs 1810. The name of the athlete is also displayed at 1825, along with a drop down menu 1826 for selection of different sports applications, the example shown being “100 m Sprint AI”. Further, there is a button 1827 to activate the analysis using a “selected AI engine” and an associated progress bar 1828 showing the progress of analysis. Finally, there is displayed options in the form of selectable icons for the processed video within a panel 1830, those being (respectively from left to right in panel 1830): to view the video; to download the video; to convert the video to JSON format; and to compare the video to another existing video. Further, there is provided a search functionality shown as search field 1831, a filter button 1832 to enable filtering of results by library name, AI engine, athlete name, amongst others. There is also a profile link 1835 where profile settings can be viewed and changed, such as password, other profile details, payments for use of service, and the option to log out of the present profile.

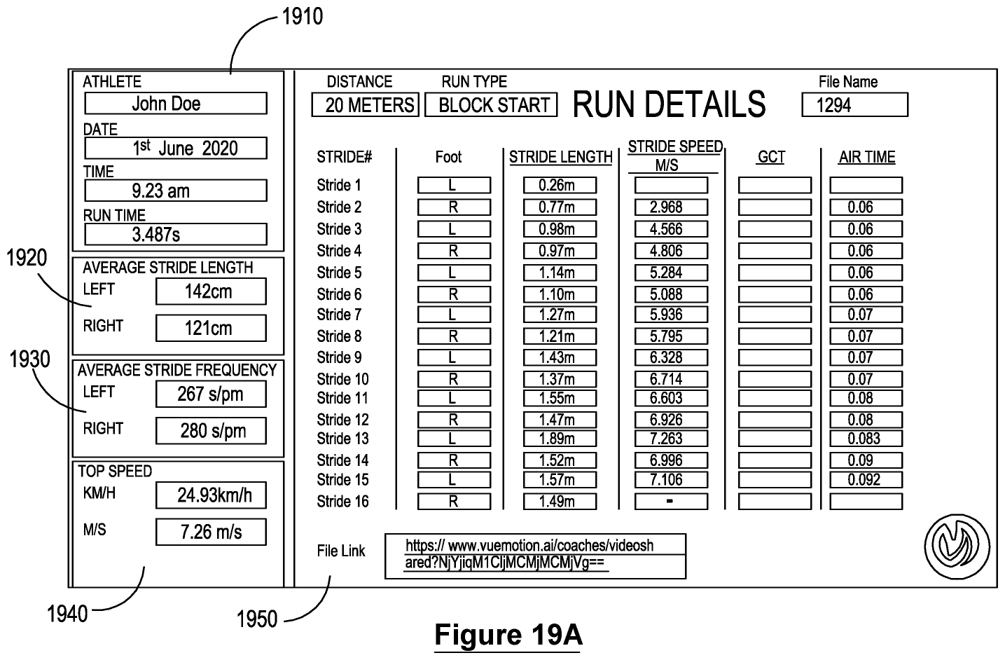

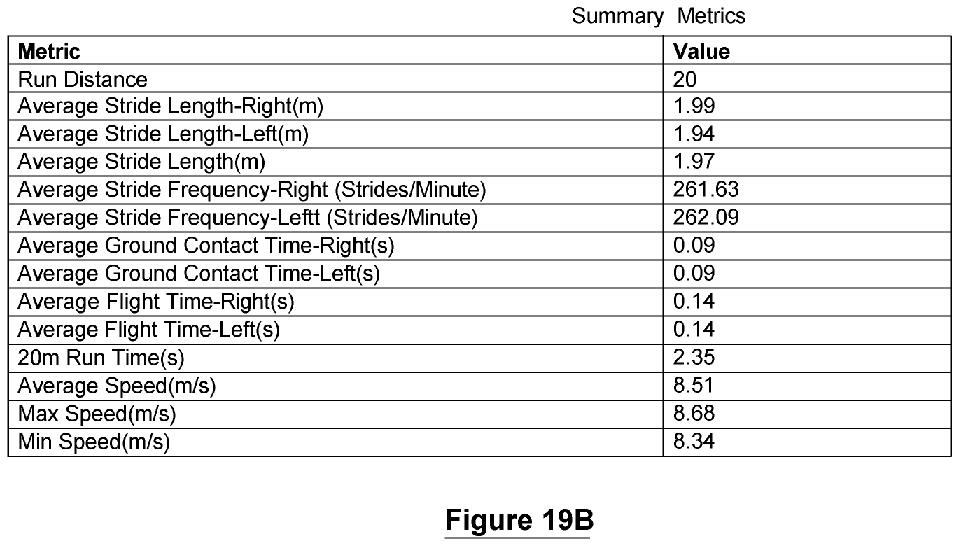

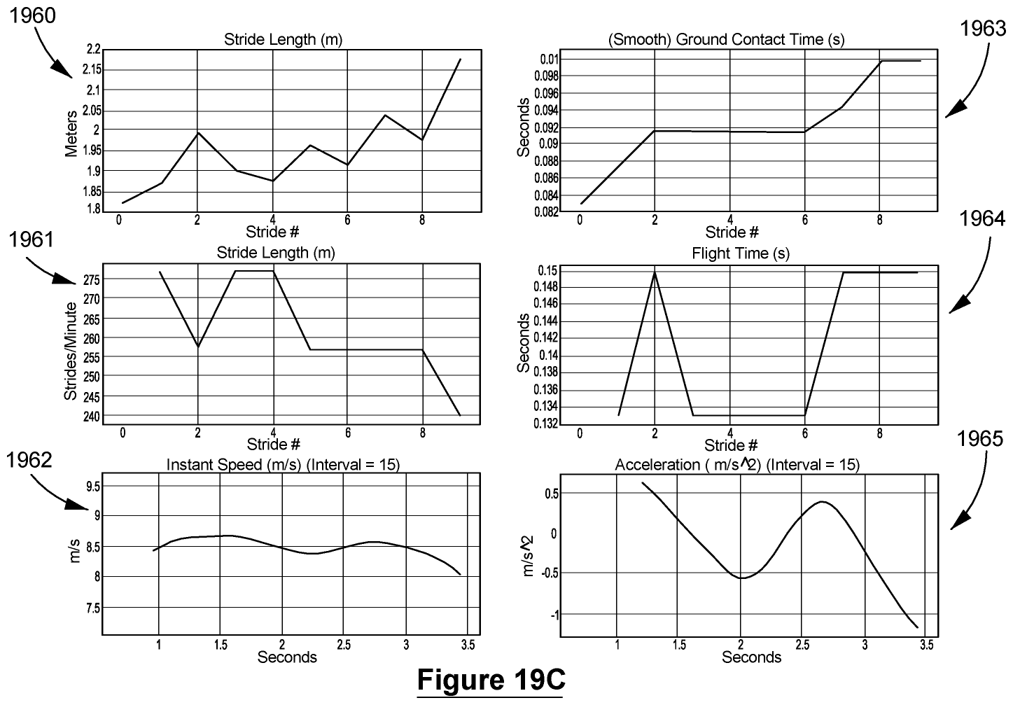

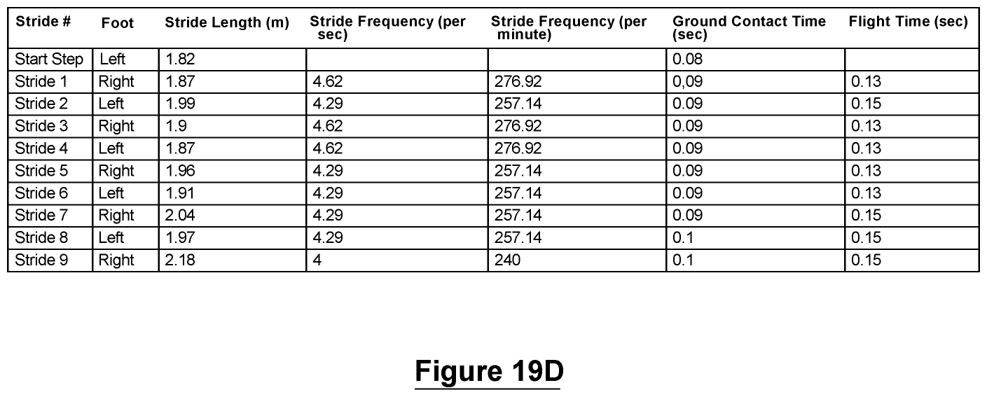

Referring to FIG. 19A, an embodiment of displayed performance metrics take the form of an athlete dashboard containing a plurality panels each providing a plurality of numerical metrics. A first panel 1910 contains fields for the following:
Athlete name, with “John Doe” as the example shown.
Date of the visual data capture, with “1^{st} June 2020” as the example shown.
Time of the visual data capture, with “9:23 am” as the example shown.
Run time of subject 102 in the 20m interval, with “3.487 s” as the example shown.

A second panel 1920 contains average stride length fields including:
Left average stride length, with “142 cm” as the example shown.
Right average stride length, with “121 cm” as the example shown.

A third panel 1930 contains average stride frequency fields including:
Left average stride frequency, with “267” strides per minute as the example shown.
Right average stride frequency, with “280” strides per minute as the example shown.

A fourth panel 1940 contains top speed fields including:
Top speed in kilometres per hour, with “24.93 km/h” as the example shown.
Top speed in metres per second, with “7.26 m/s” as the example shown.

A fifth panel 1950 contains more low level run details, including:
Distance of the run (labelled “DISTANCE”), with “20 METRES” as the example shown.
Type of Run (labelled “RUN TYPE”), with “BLOCK START” as the example shown.
File name of the specific run for that athlete, with “1294” as the example shown.
A plurality of columns including:
   A first column entitled “Stride #” with each stride provided with a label, that being “Stride 1”, “Stride 2”, . . . , “Stride 16” as the examples shown.
   A second column entitled “Foot” with each labelled stride from the first column denoted as a left or right foot stride, with the relevant stride denoted as “L” for left foot or “R” for right foot.
   A third column entitled “STRIDE LENGTH” with each labelled stride from the first column having an associated length, with stride 1 as “0.26m”, stride 2 as “0.77m”, stride 3 as “0.98”, . . . etc. as shown.
   A fourth column entitled “GCT” with each labelled stride from the first column having an associated ground contact time.
   A fifth column entitled “Air Time” with each labelled stride from the first column having an associated air time, where the athlete is not touching the ground.
A URL link to the video file of the run (labelled “File Link”), with an example URL shown.

Referring to FIG. 19B, an embodiment of displayed performance metrics take the form of summary metric table with left and right columns respectively providing a metric name (the “Metric” column) and a numerical metric value (the “Value” column). In this example, the following metrics and values are illustrated: a “Run Distance” metric having a value of “20” indicating 20 metres; an “Average Stride Length-Right (m)” metric having a value of “1.99” indicating 1.99 metres; an “Average Stride Length-Left (m)” metric having a value of “1.94” indicating 1.94 metres; an “Average Stride Length (m)” metric having a value of “1.97” indicating 1.97 metres; an “Average Stride Frequency-Right (Strides/Minute)” metric having a value of “261.63” indicating a frequency of 261.63 strides per minute; an “Average Stride Frequency-Left (Strides/Minute)” metric having a value of “262.09” indicating a frequency of 262.09 strides per minute; an “Average Ground Contact Time-Right(s)” metric having a value of “0.09” indicating 0.09 seconds; an “Average Ground Contact Time-Left(s)” metric having a value of “0.09” indicating 0.09 seconds; an “Average Flight Time-Right(s)” metric having a value of “0.14” indicating 0.14 seconds; an “Average Flight Time-Left(s)” metric having a value of “0.14” indicating 0.14 seconds; a “20m Run Time(s)” metric having a value of “2.35” indicating 2.35 seconds; an “Average Speed (m/s)” metric having a value of “8.51” indicating 8.51 metres per second; a “Max Speed (m/s)” metric having a value of “8.68” indicating 8.68 metres per second; and a “Min Speed (m/s)” metric having a value of “8.34” indicating 8.34 metres per second.

Referring to FIG. 19C, an embodiment of displayed performance metrics take the form of a plurality of line graphs, six example being shown as follows: · A stride length graph plotting consecutive strides in metres, denoted by reference 1960;
A stride frequency graph plotting stride frequencies for consecutive strides in metres per second, denoted by reference 1961;
An instance speed graph, similar to that of FIG. 14D, plotting instant speed in metres per second over a time period, denoted by reference 1962;
A ground contact time graph plotting ground contact times for consecutive strides in seconds, denoted by reference 1963;
A flight time graph plotting flight times for consecutive strides in seconds, denoted by reference 1964; and
An acceleration graph plotting acceleration in metres per second squared over a time period, denoted by reference 1965.

Referring to FIG. 19D, an embodiment of displayed performance metrics take the form of step by step analysis table (similar to the table of panel 1950) having a plurality of columns including:
A first column entitled “Stride #” with each stride provided with a label, that being “Start Step”, “Stride 1”, “Stride 2”, . . . , “Stride 9” as the examples shown.
A second column entitled “Foot” with each labelled stride from the first column denoted as a left or right foot stride, with the relevant stride denoted as “Left” for left foot or “Right” for right foot.
A third column entitled “Stride Length (m)” with each labelled stride from the first column having an associated length in metres, with start step as “1.82”, with stride 1 as “1.87”, stride 2 as “1.99”, stride 3 as “1.9”, . . . etc. as shown.
A fourth column entitled “Stride Frequency (per second)” with each labelled stride from the first column having an associated stride frequency per second, with stride 1 as “4.62”, stride 2 as “4.29”, stride 3 as “4.62”, . . . etc. as shown.
A fifth column entitled “Stride Frequency (per minute)” with each labelled stride from the first column having an associated stride frequency per minute, with stride 1 as “276.92”, stride 2 as “257.14”, stride 3 as “276.92”, . . . etc. as shown.
A sixth column entitled “Ground Contact Time (sec)” with each labelled stride from the first column having an associated ground contact time in seconds.
A seventh column entitled “Flight Time (sec)” with each labelled stride from the first column having an associated flight time in seconds (also referred to as “air time”), where the athlete is not touching the ground.

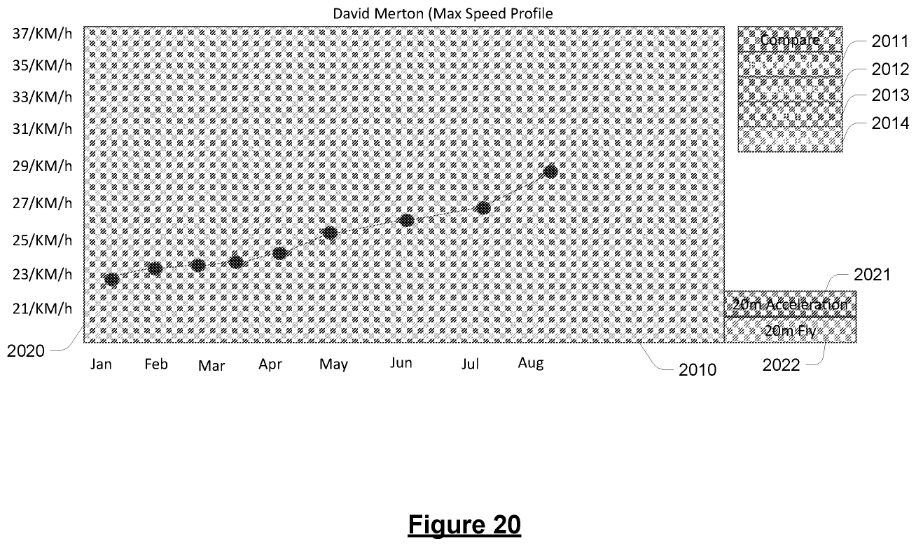

Referring to FIG. 20, an embodiment of displayed performance metrics take the form of a line graph showing the top speed of an athlete over the course of a number of months. The example graph, for an athlete named “David Merton” shows a series of plots for each of the months of January (“Jan”) through to August (“Aug”) on an x axis 2010 and kilometres per hour (from 21 km/h to 37 km/h) on a y axis 2020. The units of x axis 2010 of the graph can be selected from the following selectable “Compare” options:
“Fastest Runs” 2011 where the tops speeds of the fastest runs of the athlete are plotted. This will consist of the athletes top 10 runs (which will obviously be contingent on the number of runs the athlete has recorded, for example, if an athlete has only recorded 5 runs, only those 5 will be plotted), or in other embodiments, a different number of runs.
“Sessions” 2012 where top speed of runs of a certain training session will be plotted.
“Years” 2013 where the top speed of a calendar year will be plotted.
“Months” 2014, which is displayed, where the top speed of a month will be plotted.

The results plotted can also be toggled between “20m Acceleration” category runs or “20m Fly” category runs using buttons 2021 and 2022, respectively.

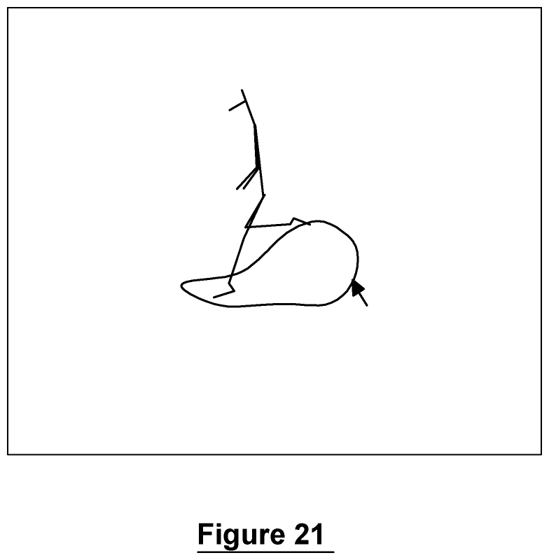

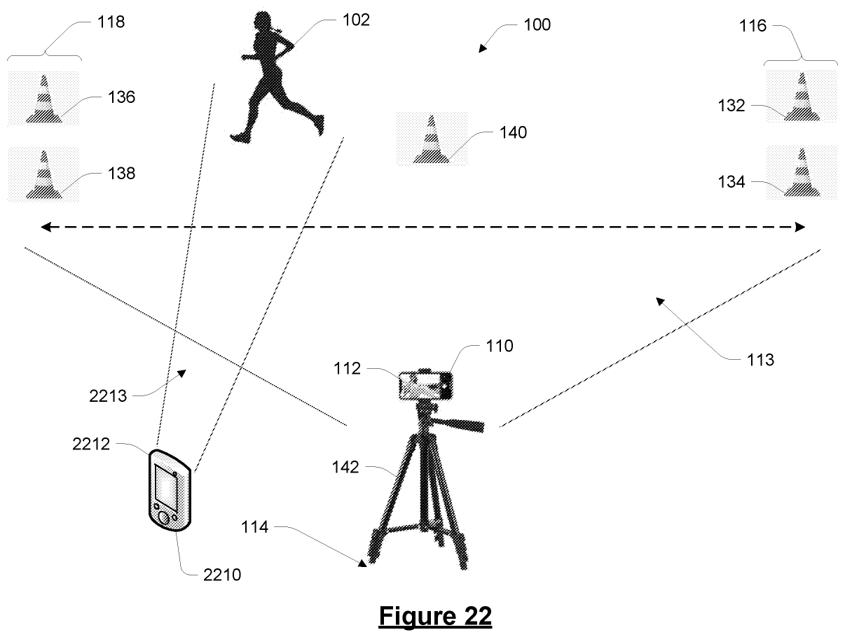

Referring to FIG. 21, an embodiment of displayed performance metrics takes the form of a result of cycle analysis of a limb of subject 102. Such cycle analysis involves placing each image frame of subject 104 in motion at one location so movement is essentially viewed without the general location of subject 102 changing. That is, a centre point of subject 102, such as the centre of the hips, is stationary whilst the rest of subject 102 moves in a running motion relative to that centre point. Some alignment and resizing may be used so that the captured visual data is consistently aligned and sized. A pose detection point (or points) such as one foot of subject 102 is then selected and the trajectory of the movement of that foot is captured to form a complete shape from the start of the cycle to the end of each cycle. Such analysis and results can be used to analyse foot dorsi flexion and plantar flexion throughout a cycle of subject 102 over the 20m interval as well as frontside and backside mechanics of the motion of subject 102. It will be appreciated that in other embodiments, motion cycles of other human pose points of subject 102 are utilised.

System 100 will also provide other forms of feedback based on the analysis of the captured visual data. Such feedback, in various embodiments, is provided on smartphone 110 (or a subsystem as a visual display) as lights, an audible tone from a speaker, a voice or other sound from the speaker and/or a vibration where the outputted feedback is relevant to the user for the purpose of identifying changes or thresholds.

An example of outputting audible feedback is outputting an audible beat to drive stride frequency. The frequency of the beat is based on a running profile of subject 102. For example, if a subject has a recorded maximum stride frequency of 270 strides per minute:
Option 1: output to the subject whilst moving (through headphones or a speaker), a predetermined beat set at 280 strides per minute with the subject attempting to run at the higher stride frequency in time with the beat being played; and
Option 2: output to the subject a predetermined beat set at 280 strides per minute prior to running, with the subject attempting to recreate this stride frequency, using AI analysis to capture stride frequency and determine result.

The subject can then be retested through system 100 to measure stride frequency following the use of the above options and the performance before and after the audible feedback can be compared using the performance metrics described herein.

Generally speaking, all performance metrics, data and associated video and feedback is preferably automatically (but otherwise manually) associated with an athlete profile of a subject using one or more of the biometric identification methods recited above, or manual input, to be used as a basis of measurement at the time. The captured data can not only be compared to past performance of the same subject over time, but also another different subject, for example two different runners, in a manner similar to the overlying metric illustrated in FIG. 8. Instead of the overlay being purely for the one subject, a comparative performance metric is formed comparing the form of two different subjects (having their own respective athlete profiles). In addition to the hip normalisation that is required for a single subject, a height normalisation process will also be required as the respective heights of the two subjects will be different. Height normalisation would scale the respective heights of the two subjects such that they are displayed as if they are the same height, for example a 1 metre proportional representation model for the purpose of comparison. Use of height normalisation and proportional representation model is used to visually compare two instances of the same subject as well as two different subjects. Such a normalisation process provides a more meaningful overlayed comparison metric than would be produced if the overlayed representations were shown at their respective different heights. Further, after an athlete performance is compared with another athlete, suggestive analysis notes are generated for the athlete based on the comparison. For example, an “ideal” biomechanical model can be generated using models of the fastest athlete of same physical characteristics. Further, a visual comparison of a subject's biomechanical model with such an “ideal” enables improvement feedback to be provided, for example, straightening left leg further.

Further, a comparison of each stride of a single captured run can occur, with the form of each stride being compared. The following performance metrics in relation to stride are determined by system 100:
Average stride length, which is broken down into the following further detailed metrics:
   Stride length as a factor of trochanter length.
   Optimum stride length, that being 2.35 times greater trochanter length (for females) and 2.43 times greater trochanter length (for males).
   At on maximum velocity performance.
   Calculated stride length compared to optimum stride length, with the difference being highlighted.
Left leg versus right leg stride length caparison.
Average stride frequency.
Left leg versus right leg stride frequency balance.
Other left leg versus right leg analysis, including the following further detailed metrics:
   Motion analysis (left leg versus right leg stride length, kleft leg versus right leg stride frequency).
   Hand motion analysis—left hand versus right hand.
   Knee motion analysis—looking at knee height, for example, a height comparison of left knee and right knee in respect of hip point.

Further, calculated ‘scores’ for key aspects of athlete performance are displayed plus theoretical scores based on improvement in aspects of athlete performance. Such scores are calculated based on the above performance metrics along with general comparative factors such as symmetry of running form (that is, a higher score for greater symmetry), and consistency of running form (that is, a higher score for the running form of an athlete being consistent across a single run or based on historical captured data).

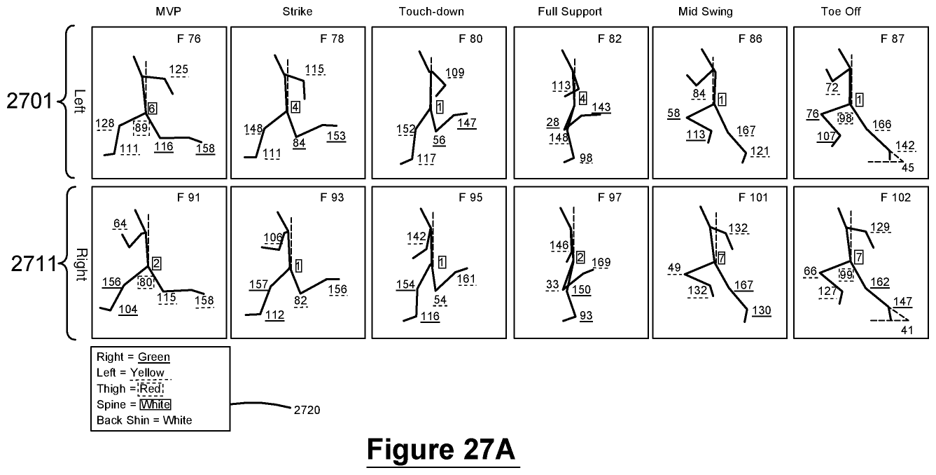

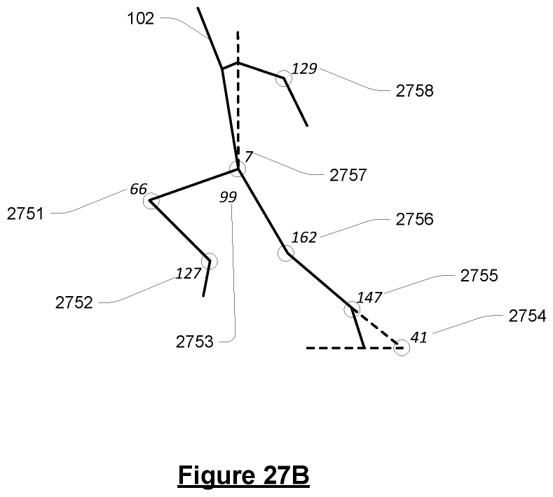

System 100 can be further utilised to identify, extract, organise and analyse various points of a step cycle during an acceleration motion and a running motion, amongst others, of subject 102. This is done using the above described video capture and analysis techniques, including steps 1201 to 1207 of process 1200, to produce a “kinogram”—which is essentially a series of related estimated human pose models for a subject such as those illustrated in FIG. 7B, FIG. 7C, FIG. 8, FIG. 26 and FIG. 27A.

Server 120 identifies from the captured video, the position of the lower limbs of subject 102 during the gait cycle whilst accelerating or running. According to the above described techniques, subject 102 moving through field of vision 113 of camera 112 and being captured on video are identified as a human and a human pose estimation model is used to map various anatomical landmarks of the body. Angles are applied to the joints captured in the estimated human pose model and are then extracted into a kinogram. Additional angles are applied to subject 102 including an angle of lean from a vertical line drawn at the hip (shown in FIGS. 26 and 27 on each estimated human pose model as a vertical dotted line) and the back shin angle at toe off (shown in FIGS. 26 and 27 on each estimated human pose model of set 2601 and on the toe off estimated human pose models of set 2701 and set 2711 as a dotted line extending from a lower rear leg extending to intersect with a horizontal dotted line illustrating an angle from the horizontal of the lower rear leg).

Two kinogram types are illustrated in FIGS. 26 and 27, respectively related to an acceleration motion and an upright running motion of subject 102.

Referring to FIG. 26, there is illustrated two sets of estimated human pose models 2601 and 2611, similar to the estimated human pose models of FIG. 7B but illustrating seven consecutive instances of the same phase during an acceleration motion of subject 102.

Set 2601 shows subject 102 captured in seven consecutive toe off phases shown during an acceleration motion moving right to left. Thus, each phase is labelled, from right to left, Step 1 to Step 7, and each individual phase is also labelled with the frame number (for example, F 98 for frame 98, F 114 for frame 144, etc.) Toe off provides angles of ankle, knees, hips and spine as well as back shin angle from the ground.

Set 2611 shows subject 102 captured in seven consecutive touch down phases shown during an acceleration motion moving right to left. Thus, as with set 2601, each phase is labelled, from right to left, Step 1 to Step 7, and each individual phase is also labelled with the frame number (for example, F 102 for frame 102, F 118 for frame 148, etc.) Touch down replaces the back shin angle from the ground at toe off with the foot placement at the toe on the ground in relation to the hip.

The acceleration model identifies and extracts the first 7 steps during the acceleration of subject 102. This includes toe off and touch down phases, which is, respectively, the frame when the toe of subject 102 is last on the ground (set 2601) and the frame where the toe of subject 102 touches the ground (set 2611).

This kinogram type related to acceleration motion enables comparison and subsequent analysis of the same phase position of subject 102 during acceleration.

It will be appreciated that in other embodiments, the instances of the same phases will not be consecutive (for example, every two cycles is modelled rather than every cycle). In yet other embodiments, phases other than toe off and touch down are modelled.

Referring to FIG. 27A, there are is illustrated two sets of estimated human pose model phases 2701 and 2711, similar to the estimated human pose model phases of FIG. 7B, illustrating six estimated human pose model phases where subject 102 (as labelled on FIG. 27, as respectively shown from left to right) is in the maximum vertical projection (MVP), strike, touch down, full support, mid swing and toe off phases during an upright run motion moving left to right.

Set 2701 shows left side body movement of subject 102 captured in the six phases shown, focusing on the left foot, leg and arm. Each individual phase is also labelled with the frame number (for example, F 59 for frame 59, F 61 for frame 61, etc.)

Set 2711 shows right side body movement of subject 102 captured in the six phases shown, focusing on the right foot, leg and arm. Each individual phase is also labelled with the frame number (for example, F 73 for frame 73, F 75 for frame 75, etc.)

A legend explaining the various anatomical features of each estimated human pose model phase is provided, denoted by reference 2720.

The key positions for kinograms for a running motion, as noted above, are:
“Maximum Vertical Projection (MVP)” which is the maximal height of vertical projection, as defined by the frame where the position of the hip of subject 102 at its highest point or where both feet of subject 102 are parallel to the ground.
“Strike” which is the frame where the position at which the swing-leg hamstring of subject 102 is under maximal stretch.
“Touch Down” which is the first frame where the foot of the retracting swing leg of subject 102 contacts the ground.
“Full Support” which is the frame where the midfoot of subject 102 is directly under the pelvis of subject 102.
“Mid Swing” which is the frame where the heel of the swing leg of subject 102 is directly under the centre of the hip of subject 102.
“Toe Off” which is the last frame where the rear foot of subject 102 is leaving the ground.

This kinogram type related to upright run motion enables comparison and subsequent analysis of the key phase positions for right and left side of subject 102 during an upright run.

Referring to FIG. 27B, the detail of the toe off phase pose of set 2711 can be more clearly seen. In particular, examples of the following joint angles are provided:
The angle of the left (front) knee, denoted by reference 2751, is shown as 66 degrees.
The angle of the left (front) ankle, denoted by reference 2752, is shown as 127 degrees.
The thigh split angle from hip to front knee and back knee, denoted by reference 2753, is shown as 99 degrees.
The right (back) shin angle with respect to the horizontal ground line, denoted by reference 2754, is shown as 41 degrees.
The angle of the right (rear) ankle, denoted by reference 2755, is shown as 147 degrees.
The angle of the right (rear) knee, denoted by reference 2756, is shown as 162 degrees.
The spine lean angle respect to the vertical line, denoted by reference 2757, is shown as 7 degrees.
The angle of the right elbow, denoted by reference 2758, is shown as 129 degrees.

The key positions in particular for toe off and touch down are determined as set out above as well as using the following techniques:
A Deep learning method and model trained into system 100. In one embodiment a long short-term memory (LSTM) based deep learning method is used. The deep learning model takes normalized pose points as inputs and generates a probabilistic output if a frame is a toe off or touch down or neither.
The probabilistic values for multiple frames are smoothed and a peak value is found to determine toe off/touch down frames.

For frames between consecutive toe off and touch down frames, other key events are identified by evaluating the pose points as follows:
For MVP, the maximal height of vertical projection is defined by the position of the hip of subject 102 at its highest point or where both feet are parallel to the ground. A method to detect an MVP frame uses knee and ankle pose points to determine that these points are vertically parallel.
For Strike, where the swing-leg hamstring is under maximal stretch, empirical relationships are identified for humans running as captured at set frame rate. For example, at 240 fps, a Strike frame is approximately 8 frames after an MVP frame.
For Full Support, the frame where the midfoot is directly under the pelvis is taken as when the hip of subject 102 and the heel point of the leg of subject 102 touching the ground are in a straight line.
For Mid Swing, where the heel of the swing leg is directly under the centre of the hip, is the frame where the heel point of the swing leg of subject 102 is in front of the knee of the supporting leg of subject 102 and the hip point and the heel point of the swing leg of subject 102 are almost aligned in a vertical straight line.

In summary, the steps for representing kinematic information of a subject in motion are as follows:
Identifying a human subject in a scene;
Identifying key anatomical points in the human body of the subject;
Identifying the type of motion as one of predetermined types;
Visually representing the motion as a sequence of one or more key events of the running kinematics; and
Representing a kinematic profile (that is, a kinogram) as a sequence of numbers representing the key relationships between different body parts of the person.

Kinograms can be defined as specific instances over a motion of a run at pre-determined angles. For running kinematics the angles are applied to the joints captured and then are extracted into a kinogram. Additional angles are then applied to the human subject such as the above noted angle of lean and back shin angle at toe off.

Two kinematic profiles are also able to be compared using kinograms. In one embodiment, Euclidean distance between key pose angles is estimated for each phase of kinograms. The sum of distance is used as a metric for comparison. In another embodiment, a normalized difference is used.

Whilst the embodiments described above are in respect of a human subject, it will be appreciated that many aspects of this technology is able to be used for analysis of movement through an interval of non-human subjects, for example animals such as dogs and horses.

### Advantages of Detailed Embodiments

It will be appreciated that the embodiments of system 100 described herein are advantageous over known systems as it has been devised to address the limitations of known systems. More specifically, system 100 achieves the following advantages:
The solution allows a user capture video footage of themselves or another person through a smart phone application or be able to upload to a video management software environment on device and in a cloud environment.
Based on the results of kinematic data captured, feedback is provided instantly as an overlay or processed and provided to a user as an overlay or metric at a later time for further analysis and action.
Allows for use of one or more standard smartphone cameras, rather than requiring any specific specialised equipment.
Allows the capture of requisite visual data without the use of specific wearable products or garments on the subject.
From the captured visual data, achieving automatic identification of relevant physical markers and human pose points.
Stride-by-stride performance analysis of subject through the generated performance metrics.
User friendly representations, such as a shoe in place for each stride.
Utilising unique human pose point identification using the triple point diagonal cross with center line method in order to more clearly analyse 3D rotational movement of limbs and joints.
Contextualising athlete key performance statistics through the performance metrics in order to suggest actions to improve future performance. Feedback provided to the subject is relevant for the purpose of identifying changes and/or thresholds.
All metrics, data and associated video and feedback is automatically, or manually mapped back to an individual subject using biometric characteristics of gait, facial recognition or manual input, to be used as a basis of measurement at the time.
The visual data captured can be compared to the same athlete over time or another athlete.
A computer vision application allows data and visualisations to be added to the video in different forms for analysis.
The use of a second camera focused on subject 102 for improves signal to noise ratio.
Noise cancellation to detect and counter errors in estimating human pose points.
The capability of detecting multiple subjects in the same captured visual data and perform analysis on each of the detected subjects.

As such, system 100 provides a means to capture and analyse the movement of a subject in a convenient way such that only standard equipment is required, and in an informative way through the biomechanical models formed and the performance metrics generated.

### Conclusions and Interpretation

Throughout this specification, where used, the terms “element” and “component” are intended to mean either a single unitary component or a collection of components that combine to perform a specific function or purpose.

It should be appreciated that in the above description of exemplary embodiments of the invention, various features of the invention are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, electrical or optical contact, or that two or more elements are not in direct contact with each other but yet still co-operate or interact with each other.

Unless specifically stated otherwise, as apparent from the following discussions, it is appreciated that throughout the specification discussions utilizing terms such as “processing,” “computing,” “calculating,” “determining”, analysing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, for example, from registers and/or memory to transform that electronic data into other electronic data that, for example, may be stored in registers and/or memory. A “computer” or a “computing machine” or a “computing platform” may include one or more processors.

Some methodologies or portions of methodologies described herein are, in one embodiment, performable by one or more processors that accept computer-readable (also called machine-readable) code containing a set of instructions that when executed by one or more of the processors carry out at least one of the methods described herein. A memory subsystem of a processing system includes a computer-readable carrier medium that carries computer-readable code (for example, software) including a set of instructions to cause performing, when executed by one or more processors, one of more of the methods described herein. Note that when the method includes several elements, for example, several steps, no ordering of such elements is implied, unless specifically stated. The software may reside in the storage medium, or may also reside, completely or at least partially, within the RAM and/or within the processor during execution thereof by the computer system. Thus, the memory and the processor also constitute computer-readable carrier medium carrying computer-readable code.

Furthermore, a computer-readable carrier medium may form, or be included in a computer program product.

In alternative embodiments, unless otherwise specified, the one or more processors operate as a standalone device or may be connected, for example, networked to other processor(s), in a networked deployment, the one or more processors may operate in the capacity of a server or a user machine in server-user network environment, or as a peer machine in a peer-to-peer or distributed network environment. The one or more processors may form a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine.

Note that while only a single processor and a single memory that carries the computer-readable code may be shown herein, those in the art will understand that many of the components described above are included, but not explicitly shown or described in order not to obscure the inventive aspect. For example, while only a single machine is illustrated, the term “machine” shall also be taken to include any collection of machines that individually or jointly execute a set (or multiple sets) of instructions to perform any one or more of the methodologies discussed herein, unless otherwise specified.

Thus, one embodiment of each of the methods described herein is in the form of a computer-readable carrier medium carrying a set of instructions, for example, a computer program that is for execution on one or more processors, for example, one or more processors that are part of web server arrangement. Thus, as will be appreciated by those skilled in the art, embodiments of the present invention may be embodied as a method, an apparatus such as a special purpose apparatus, an apparatus such as a data processing system, or a computer-readable carrier medium, for example, a computer program product. The computer-readable carrier medium carries computer readable code including a set of instructions that when executed on one or more processors cause the processor or processors to implement a method. Accordingly, aspects of the present invention may take the form of a method, an entirely hardware embodiment, an entirely software embodiment or an embodiment combining software and hardware aspects. Furthermore, the present invention may take the form of carrier medium (for example, a computer program product on a computer-readable storage medium) carrying computer-readable program code embodied in the medium.

The software may further be transmitted or received over a network via a network interface device. While the carrier medium may be shown in an embodiment to be a single medium, the term “carrier medium” should be taken to include a single medium or multiple media (for example, a centralized or distributed database, and/or associated caches and servers) that store the one or more sets of instructions. The term “carrier medium” shall also be taken to include any medium that is capable of storing, encoding or carrying a set of instructions for execution by one or more of the processors and that cause the one or more processors to perform any one or more of the methodologies of the present invention. A carrier medium may take many forms, including but not limited to, non-volatile media, volatile media, and transmission media. Non-volatile media includes, for example, optical, magnetic disks, and magneto-optical disks. Volatile media includes dynamic memory, such as main memory. Transmission media includes coaxial cables, copper wire and fibre optics, including the wires that comprise a bus subsystem. Transmission media also may also take the form of acoustic or light waves, such as those generated during radio wave and infrared data communications. For example, the term “carrier medium” shall accordingly be taken to included, but not be limited to, solid-state memories, a computer product embodied in optical and magnetic media; a medium bearing a propagated signal detectable by at least one processor of one or more processors and representing a set of instructions that, when executed, implement a method; and a transmission medium in a network bearing a propagated signal detectable by at least one processor of the one or more processors and representing the set of instructions.

It will be understood that the steps of methods discussed are performed in one embodiment by an appropriate processor (or processors) of a processing (i.e., computer) system executing instructions (computer-readable code) stored in storage.

### INDUSTRIAL APPLICABILITY

The arrangements described are applicable to the sporting industry and, particularly to systems and devices for performance tracking and analysis of runners. Therefore, the invention is clearly industrially applicable.

1. A method for generating a biomechanical model of a subject in motion including the steps of:
capturing, by a single supported motion capture device from a capture position, visual data of the subject as it moves between two distance markers that are disposed at a distance apart from each other and in a field of vision of the motion capture device;
from the captured visual data, recognising human pose points on the subject to extract kinematic data of the subject; and
based on the extracted kinematic data, recognising a plurality of predefined anatomical components of the subject to construct the biomechanical model of the subject.

2. A method according to claim 1 wherein the captured visual data includes a plurality of video frames, and the constructed biomechanical model is based on at least one frame capturing the subject in a predefined stance.

3. A method according to claim 2 wherein the predefined stance is one or more of:
a toe-off stance whereby a back foot of the subject is lifted off a ground push-off point;
a touch down stance whereby a front foot of the subject is about to contact a ground drop point; and
a full support stance whereby the front foot flattens on the ground drop point and whereby hip and heel human pose points of the subject are vertically aligned.

4. A method according to claim 1 wherein the motion capture device is substantially stationarily supported.

5. A method according to claim 1 wherein the motion capture device is a camera.

6. A method according to claim 5 wherein the camera is a smartphone camera.

7. A method according to claim 5 wherein the camera is an IP camera.

8. A method according to claim 1 wherein the motion capture device includes two synchronised cameras.

9. A method according to claim 1 wherein the visual data of a subject is captured without the use of wearable subject makers on the subject.

10. A method according to claim 1 wherein the distance between the two distance markers is a predetermined distance of 20 metres.

11. A method according to claim 1 including the further step of formulating based on the constructed biomechanical model, a motion performance metric.

12. A method according to claim 1 including the further step of outputting the motion performance metric for visual display on a display device.

13. A system for generating a biomechanical model of a subject in motion including:
a single supported motion capture device configured to capture from a capture position, visual data of a subject as it moves between two distance markers that are disposed at a distance apart from each other and in a field of vision of the motion capture device;
a central data processing server in communication with the motion capture device, the central data processing server configured to:
recognise, from the captured visual data, human pose points on the subject;
extract, from the recognised human pose points, kinematic data of the subject;
recognise, based on the extracted kinematic data, a plurality of predefined anatomical components of the subject; and
construct, based on the recognised plurality of predefined anatomical components, the biomechanical model of the subject.

14. A method according to claim 1 including the further steps of:
recognising a captured length of a component of the subject having a known real-world length;
mapping the known real-world length of the component to the captured length of the component; and
based on the biomechanical model, formulating a motion performance metric, wherein the biomechanical model includes real-world lengths based on the mapping.

15. A method according to claim 1 including the further steps of:
capturing, by the single stationarily supported motion capture device, visual data of the at least two subjects as they move between the two distance markers;
from the captured visual data, individually detecting each of the at least two subjects such that each detected subject is isolated;
from the captured visual data, for each detected subject;
recognising human pose points on the subject to extract kinematic data of the subject; and
based on the extracted kinematic data, recognising a plurality of predefined anatomical components of the subject to construct the biomechanical model of the subject.

16. A method for providing a visual comparison of a first biomechanical model and a second biomechanical model, the method including the steps of:
generating the first and second biomechanical models according to the method of claim 1;
identify corresponding central points of reference on each of the first and second biomechanical models;
scale each of the first and second biomechanical models such that relative heights of the first and second biomechanical models are identical; and
overlay the scaled first and second biomechanical models at their respective corresponding central points of reference to provide the visual comparison of the first and second biomechanical models.

17. A method for identifying a current subject based on a predefined limb length ratio of a known subject, the method including the steps of:
generating a biomechanical model of the current subject in motion according to the method of claim 1, wherein the plurality of predefined anatomical components of the current subject includes a first subject limb and a second subject limb of the subject, the first subject limb having a first limb length and the second subject limb having a second limb length;
generating a current limb length ratio based on the first limb length and the second limb length;
comparing the current limb length ratio to the predefined limb length ratio; and
if the current limb length ratio and the predefined limb length ratio substantially matches, identifying the current subject as being the known subject.
